(19) United States
(12) Patent Application Publication
(10) Pub. No.: US 2025/0381028 A1
(43) Pub. Date: Dec. 18, 2025

(54) HEART VALVE PROSTHESES, TRANSCATHETER DEVICES, AND METHODS

(71) Applicant: Medtronic, Inc., Minneapolis, MN (US)

(72) Inventors: Jake Dunlea, Galway (IE); Michael R. Brady, Belfast (GB); Florentina Ene, Galway (IE); Emmanouil Kasotakis, Galway (IE); Laoise D. Conroy, Sydney (AU)

(21) Appl. No.: 19/229,663

(22) Filed: Jun. 5, 2025

(30) Foreign Application Priority Data
Jun. 17, 2024 (GR) 20240100447

(51) Int. Cl. A61F 2/24

(52) U.S. Cl. CPC A61F 2/2418; A61F 2/2433; A61F 2/2436; A61F 2220/0041; A61F 2230/0067

(57) ABSTRACT

Heart valve prostheses comprise an expandable stent frame configured to expand from a contracted orientation to an expanded orientation. The heart valve prostheses can further comprise a valve apparatus extending past a distal end of the expandable stent frame. The valve apparatus comprise a plurality of commissure posts, each commissure post of the plurality of commissure posts comprises a first end portion attached to a distal end portion of the expandable stent frame, a second end portion attached to a distal tip of the valve apparatus at a distal end of the valve apparatus. The plurality of commissure posts converge together in a distal direction towards the distal tip. The valve apparatus further comprise a plurality of leaflets each coupled to a corresponding pair of the plurality of commissure posts.

### FIELD

The present disclosure relates generally to heart valve prostheses, transcatheter devices, and methods, and more particularly to heart valve prostheses and transcatheter devices including a valve apparatus extending past a distal end of an expandable stent frame, and methods of replacing a defective heart valve.

### BACKGROUND

A human heart includes four heart valves that determine the pathway of blood flow through the heart: the mitral valve, the tricuspid valve, the aortic valve, and the pulmonary valve. The mitral and tricuspid valves are atrio-ventricular valves, which are between the atria and the ventricles, while the aortic and pulmonary valves are semilunar valves, which are in the arteries leaving the heart. Ideally, native leaflets of a heart valve move apart from each other when the valve is in an open position and meet or “coapt” when the valve is in a closed position. Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Diseased or otherwise deficient heart valves can be repaired or replaced using a variety of different types of heart valve surgeries. One conventional technique involves an open-heart surgical approach that is conducted under general anesthesia, during which the heart is stopped, and blood flow is controlled by a heart-lung bypass machine.

More recently, minimally invasive approaches have been developed to facilitate catheter-based implantation of a prosthetic heart valve or prosthesis on the beating heart, intending to obviate the need for the use of classical sternotomy and cardiopulmonary bypass. In general terms, an expandable prosthetic valve is compressed about or within a catheter, inserted inside a body lumen of the patient, such as the femoral artery, and delivered to a desired location in the heart.

However, over time, these prosthetic heart valves may also begin to become damaged or diseased, such as for example, stenosis. Accordingly, the diseased or damaged prosthetic heart valve may need replaced. Similar to that described above, catheter-based approaches have also been used to repair or replace the damaged prosthetic heart valves.

In light of the above, a need exists for a heart valve prosthesis and/or a transcatheter device that can prevent/minimize obstruction of the coronary arteries that may be caused by leaflets of a replaced heart valve during catheter-based implantations of prosthetic heart valves.

### SUMMARY

The following presents a simplified summary of the disclosure to provide a basic understanding of some aspects described in the detailed description.

Features of the present disclosure provide a heart valve prosthesis having a valve apparatus extending past a distal end of an expandable stent frame to facilitate the replacement of a damaged and/or diseased heart valve. Providing a transcatheter device with a valve apparatus that can extend past a distal end of an expandable stent frame enables the valve apparatus to extend distally past the annulus of the defective heart valve to prevent/minimize obstructions that may be caused by the leaflets of the replaced defective heart valve.

In aspects, heart valve prostheses comprise an expandable stent frame configured to expand from a contracted orientation to an expanded orientation. The expandable stent frame comprises a proximal end portion and a distal end portion. The expandable stent frame extends from the proximal end portion to the distal end portion along an elongated axis of the expandable stent frame. The heart valve prostheses can further comprise a valve apparatus extending past a distal end of the distal end portion of the expandable stent frame. The valve apparatus comprises a plurality of commissure posts, each commissure post of the plurality of commissure posts comprising a first end portion attached to the distal end portion of the expandable stent frame, a second end portion attached to a distal tip of the valve apparatus at a distal end of the valve apparatus. The plurality of commissure posts converge together in a distal direction of the elongated axis towards the distal tip. The valve apparatus further comprises a plurality of leaflets each coupled to a corresponding pair of the plurality of commissure posts.

In further aspects, transcatheter devices comprise an outer shaft. The transcatheter devices further comprise an inner shaft extending within the outer shaft and through a distal end of the outer shaft. The transcatheter devices still further comprise an expandable stent frame configured to expand from a contracted orientation to an expanded orientation. The expandable stent frame comprises a proximal end portion and a distal end portion. The expandable stent frame defines a lumen extending from the proximal end portion to the distal end portion along an elongated axis of the expandable stent frame, and a distal segment of the inner shaft distally extends outside of the distal end of the outer shaft and through the lumen of the expandable stent frame. The transcatheter devices still further comprise a valve apparatus extending from a distal end of the distal end portion of the expandable stent frame. The valve apparatus comprises a plurality of commissure posts, each commissure post of the plurality of commissure posts comprising a first end portion attached to the distal end portion of the expandable stent frame, a second end portion attached to a distal tip of the valve apparatus at a distal end of the valve apparatus. The plurality of commissure posts converge together in a distal direction of the elongated axis towards the distal tip. The valve apparatus further comprises a plurality of leaflets each coupled to a corresponding pair of the plurality of commissure posts.

In further aspects, methods comprise replacing a heart valve with a heart valve prosthesis. The heart valve prosthesis comprises an expandable stent frame comprising a proximal end portion and a distal end portion. The expandable stent frame extends from the proximal end portion to the distal end portion along an elongated axis of the expandable stent frame. The heart valve prosthesis further comprises a valve apparatus extending from a distal end of the distal end portion of the expandable stent frame. The valve apparatus comprises a plurality of commissure posts, each commissure post of the plurality of commissure posts comprising a first end portion attached to the distal end portion of the expandable stent frame, a second end portion attached to a distal tip of the valve apparatus at a distal end of the valve apparatus. The plurality of commissure posts converge together in a distal direction of the elongated axis towards the distal tip. The valve apparatus further comprises a plurality of leaflets each coupled to a corresponding pair of the plurality of commissure posts. The method comprises distally advancing the heart valve prosthesis into the heart valve while the expandable stent frame is in a contracted orientation. The method further comprises aligning a portion of the heart valve prosthesis with a portion of the heart valve. The method still further comprise radially expanding the expandable stent frame from the contracted orientation to an expanded orientation.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 is a schematic view of an exemplary transcatheter device, in accordance with aspects of the present disclosure;

FIG. 2 is a side schematic view of an exemplary preexisting prosthetic heart valve that can be replaced utilizing the transcatheter device of FIG. 1, in accordance with aspects of the present disclosure;

FIG. 3 is a schematic cross-sectional view of the exemplary preexisting prosthetic heart valve along line 3-3 of FIG. 2 illustrating a lacerated leaflet of the preexisting prosthetic heart valve;

FIG. 4 is the schematic cross-sectional view of the exemplary preexisting prosthetic heart valve of FIG. 2 illustrating non-lacerated leaflets of the preexisting prosthetic heart valve;

FIG. 5 is a side schematic view of a heart valve prosthesis that can be utilized with the transcatheter device of FIG. 1, in accordance with aspects of the present disclosure;

FIG. 6 is a partial schematic cross-sectional view of the heart valve prosthesis along line 6-6 of FIG. 5 showing the heart valve prosthesis in an inwardly bent configuration;

FIG. 7 is the partial schematic cross-sectional view of FIG. 6 showing the heart valve prosthesis in an outwardly bent configuration;

FIG. 8 is a schematic two-dimensional view of a leaflet of the heart valve prosthesis of FIG. 5, in accordance with aspects of the present disclosure;

FIG. 9 is a bottom schematic perspective view of an expandable stent frame of the heart valve prosthesis of FIG. 5, illustrating an embodiment of a hemostatic bumper coupled to the expandable stent frame in accordance with aspects of the present disclosure;

FIG. 10 is a schematic cross-sectional view of the hemostatic bumper along line 10-10 of FIG. 9, showing the hemostatic bumper coupled to the expandable stent frame of the heart valve prosthesis of FIG. 5;

FIG. 11 is a schematic cross-sectional view along line 10-10 of FIG. 10 of the expandable stent frame of FIG. 9, showing another embodiment of a hemostatic bumper coupled to the expandable stent frame of the heart valve prosthesis of FIG. 5;

FIG. 12 is a bottom schematic perspective view of the expandable stent frame of the heart valve prosthesis of FIG. 5, illustrating another embodiment of a hemostatic bumper coupled to the expandable stent frame in accordance with aspects of the present disclosure;

FIG. 13 is a side schematic view of the expandable stent frame of the heart valve prosthesis of FIG. 5 indexing with the preexisting prosthetic heart valve of FIG. 2 in accordance with aspects of the current disclosure;

FIG. 14 schematically illustrates cross-sectional view of a distal end portion of the transcatheter device along line 14-14 of FIG. 1, showing an embodiment of the distal end portion including the heart valve prosthesis of FIG. 5;

FIG. 15 schematically illustrates a cross-sectional view of another embodiment of a distal end portion of the transcatheter device along line 14-14 of FIG. 1, showing an embodiment of the distal end portion including the heart valve prosthesis of FIG. 5;

FIG. 16 is a schematic exploded view of an embodiment of the transcatheter device in accordance with aspects of the disclosure;

FIG. 17 schematically illustrates distally advancing a heart valve prosthesis comprising an expandable stent frame in a contracted orientation into a preexisting heart valve;

FIG. 18 schematically illustrates aligning a portion of the heart valve prosthesis with the preexisting heart valve;

FIG. 19 schematically illustrates a cutaway view of the preexisting heart valve illustrating radially expanding the expandable stent frame of the heart valve prosthesis from the contracted orientation to an expanded orientation;

FIG. 20, schematically illustrates lacerating at least one leaflet of the preexisting heart valve with the transcatheter device of FIG. 1 or another transcatheter device in accordance with aspects of the present disclosure;

FIG. 21-22 schematically illustrates aligning an inflation device of the transcatheter device of FIG. 1 or another transcatheter device with the lacerated leaflets of the preexisting heart valve and inflating the inflation device to reposition the leaflets of the heart valve; and

FIG. 23 schematically illustrates proximally retracting the transcatheter device of FIG. 1 or another transcatheter device to remove the transcatheter device of FIG. 1 or another transcatheter device from the implanted heart valve prosthesis.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art.

Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein—for example up, down, right, left, front, back, top, bottom, upper, lower, etc.—are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” may be used in the following description with reference to one or more valve or stent structures of the prostheses hereof and is intended to convey that the structures are shaped or formed from a material that can be provided with a mechanical memory to return the structure from a compressed or constricted delivery configuration to an expanded deployed configuration or vice versa. Non-exhaustive exemplary self-expanding materials include stainless steel, a pseudo-elastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorbornene, trans-polyisoprene, styrene-butadiene, and polyurethane. As well poly L-D lactic copolymer, oligo caprolactone copolymer and poly cyclo-octine can be used separately or in conjunction with other shape memory polymers.

Diseases associated with heart valves (e.g., a native heart valve), such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

However, over time these heart valve prostheses may also fail and require another procedure to replace the damaged or defective heart valve. Similar to replacing a native heart valve, in order to replace a failed prosthetic heart valve, a transcatheter device including a replacement prosthesis can be employed.

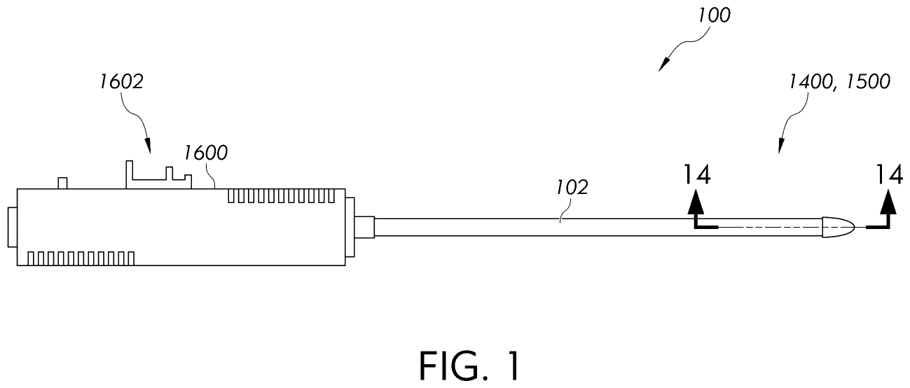

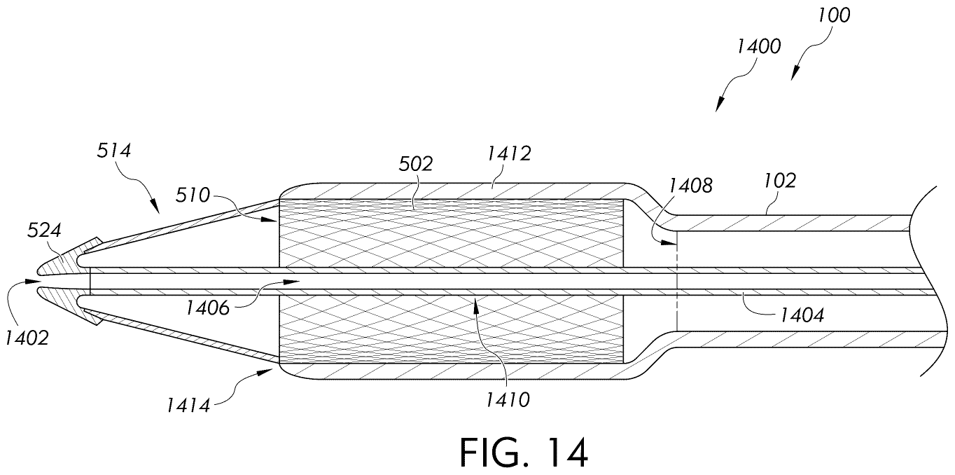

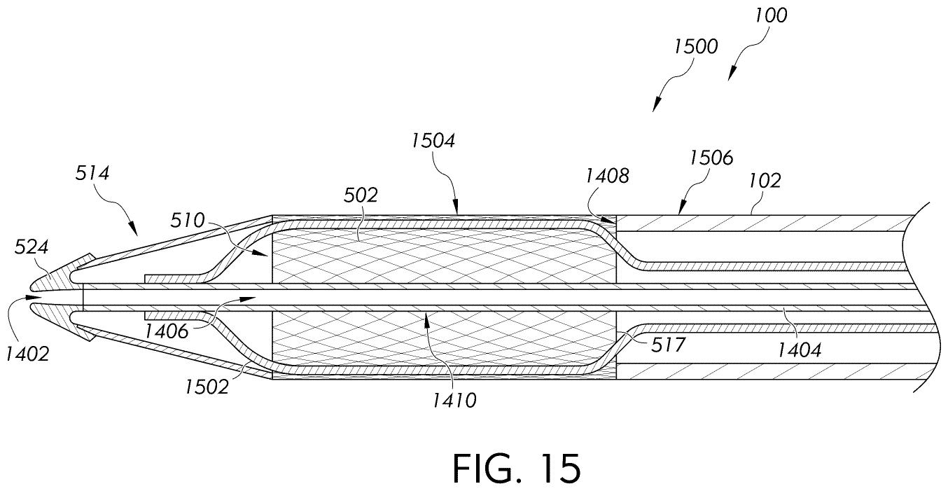

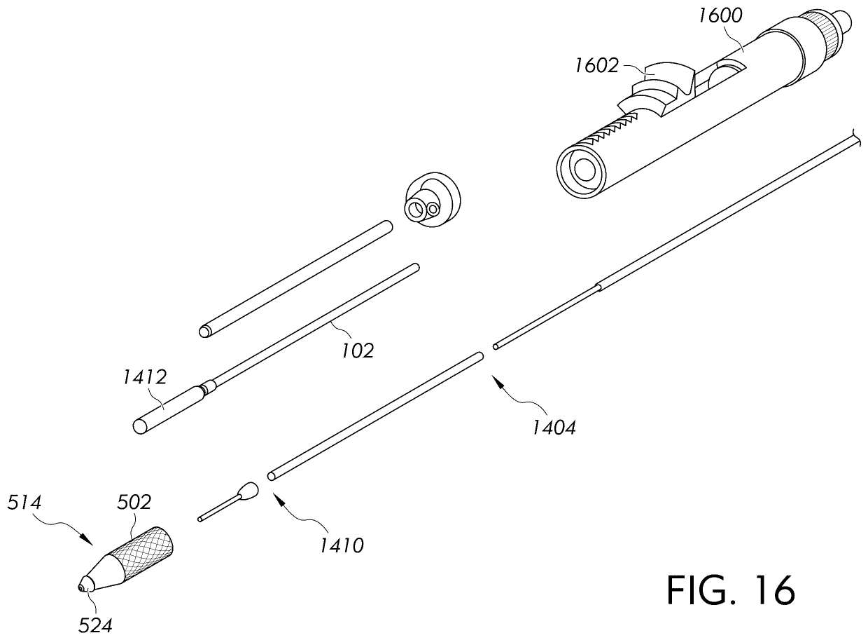

FIG. 1 schematically illustrates an exemplary transcatheter device 100 (e.g., transcatheter delivery system) in accordance with the present disclosure. As show, in aspects, the transcatheter device 100 can comprise a distal end portion 1400, 1500, an outer shaft 102, and a handle device 1600. In some aspects, described in further detail hereinafter with reference to FIG. 16, the outer shaft 102 can comprise one or more shafts (e.g., a shaft assembly). In aspects, the outer shaft 102 can couple the distal end portion 1400, 1500 (discussed with initial reference to FIGS. 14 and 15) to the handle device 1600. In further aspects, the handle device 1600 can comprise one or more actuators 1602 (i.e., one actor, two actuators, etc.) configured to actuate various components of the transcatheter device 100. For example, the handle device 1600 can be configured to proximally retract and/or distally advance the outer shaft 102. The handle device 1600 should not be construed to be limited to the above examples, and thus can interact with other components of the transcatheter device 100, which will become more apparent hereinafter.

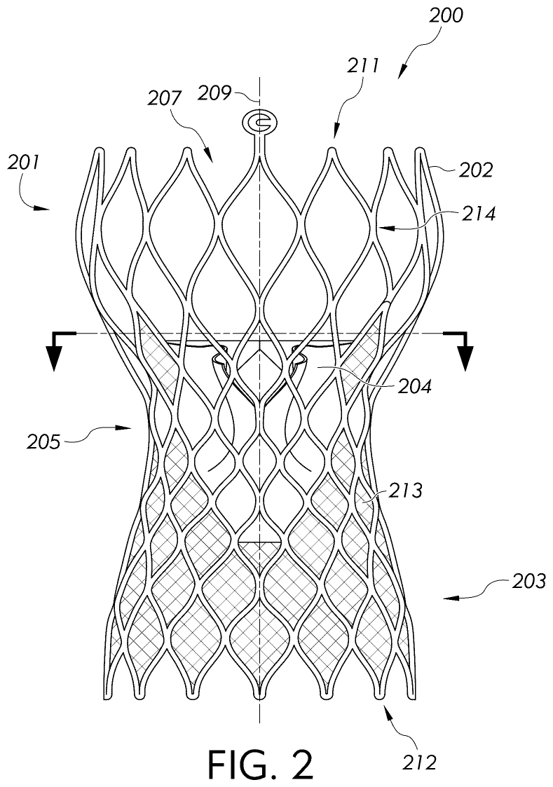

FIGS. 2-4 illustrate a preexisting prosthetic heart valve 200 that has been previously implanted within a patient and has failed, such as for example, by calcification of the preexisting prosthetic heart valve 200 resulting in a narrowing of the opening of the preexisting prosthetic heart valve 200. Accordingly, the exemplary preexisting prosthetic heart valve 200 will be utilized hereinafter with reference to aspects of the transcatheter device 100 in accordance with the present disclosure in order to described various operations and components of the transcatheter device 100.

As shown, the preexisting prosthetic heart valve 200 can comprise a stent frame 202 and prosthetic heart valve leaflets 204. In some aspects, the stent frame 202 may be a self-expandable stent frame, such as for example, a nitinol self-expanding stent frame, or may be a balloon expandable stent frame. In further aspects, the stent frame 202 can comprise a proximal portion 201 (outflow portion) comprising a proximal end 211 (outflow end) and a distal portion 203 (inflow portion) comprising a distal end 212 (inflow end). In some aspects, the proximal portion 201 and the distal portion 203 can comprise a larger diameter than an intermediate portion 205 of the stent frame 202. In some aspects, the proximal portion 201 may have a greater diameter, a smaller diameter, or an equal diameter to that of the distal portion 203. In some aspects, the stent frame 202 can comprise a lumen 207 extending through a central axis 209 of the stent frame 202.

In aspects, the proximal portion 201 can comprise a first outwardly bulging circumferential portion. The first outwardly bulging circumferential portion can encircle the lumen 207 of the stent frame 202 and may bulge radially outward with respect to the intermediate portion 205. In some aspects, the first outwardly bulging circumferential portion can taper towards the distal portion 203 and adjoin a proximal portion of the intermediate portion 205. In aspects, a distal portion of the intermediate portion 205 can adjoin the distal portion 203 of the stent frame 202. The distal portion 203 of the stent frame 202 can comprise a second outwardly bulging circumferential portion that can encircle the lumen 207 and may bulge radially outward with respect to the intermediate portion 205. In some aspects, the second outwardly bulging circumferential portion can taper towards the proximal portion 201. In aspects, as shown, the second outwardly bulging circumferential portion can have a smaller diameter than the first outwardly bulging circumferential portion. In other aspects, the second outwardly bulging circumferential portion can have a greater diameter than the first outwardly bulging circumferential portion. In yet another aspect, the second outwardly bulging circumferential portion can have a substantially equal diameter to that of the first outwardly bulging circumferential portion.

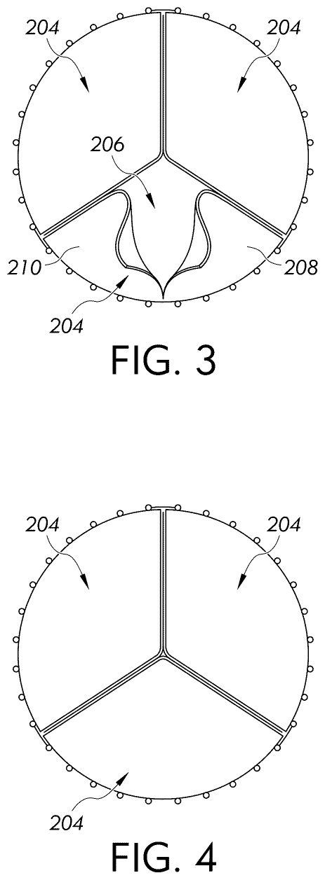

In aspects, the prosthetic heart valve leaflets 204 can comprise one or more leaflets, such as for example, a valve with three leaflets (shown in FIGS. 3 and 4). In some aspects, the prosthetic heart valve leaflets 204 can comprise a valve with two leaflets. As illustrated in FIGS. 2-3, one or more prosthetic heart valve leaflets 204 of the preexisting prosthetic heart valve 200 may be lacerated (e.g., by a laceration device). In some non-limiting examples, the prosthetic heart valve leaflets 204 can be lacerated to bisect (e.g., lacerated to divide the prosthetic heart valve leaflets 204 into two substantially equal parts) one or more of the prosthetic heart valve leaflets 204. For example, FIG. 3 shows a lacerated portion 206 of one of the prosthetic heart valve leaflets 204 that has been lacerated to bisect the prosthetic heart valve leaflets 204. Although only one prosthetic heart valve leaflet 204 is illustrated as radially lacerated (e.g., symmetrically bifurcated into two identical parts), in further embodiments, a plurality (e.g., two) or all of the prosthetic heart valve leaflets 204 may be lacerated in further embodiments.

In aspects, the stent frame 202 can comprise a plurality of cell structures 214. In aspects, the plurality of cell structures 214 can comprise diamond-shaped cell structures. However, it should be understood that the stent frame 202 is merely exemplary and any other suitable expandable stent frames may be utilized with the preexisting prosthetic heart valve 200. Furthermore, the plurality of cell structures 214 should not be limited to diamond-shaped cell structures and should not be limited to the number of cell structures and/or size of the cell structures shown in FIG. 2. Accordingly, more or less suitable cell structures with a variety of different shapes and sizes can be employed with the preexisting prosthetic heart valve 200.

As shown in FIG. 3, the lacerated portion 206 can comprise a first lacerated portion 208 and a second lacerated portion 210 that are substantially equal in size. In other aspects, various other locations along one or more of the prosthetic heart valve leaflets 204 can be lacerated such that the first lacerated portion 208 and the second lacerated portion 210 are not substantially equal. In other aspects, the prosthetic heart valve leaflets 204 can be lacerated to form more than two lacerated pieces. It should be understood that while FIG. 3 only shows one of the three prosthetic heart valve leaflets 204 lacerated, the other two leaflets can be lacerated as described above. The leaflets can be lacerated by scalpel, electrode, or other cutting techniques. It should be further understood that FIG. 3 is merely exemplary and should not limit the transcatheter device 100 to use with only the lacerated leaflets as described above. Thus, the transcatheter device 100 can be employed with other types of lacerated leaflets.

In some aspects, the prosthetic heart valve leaflets 204 may be lacerated prior to inserting the transcatheter device 100 or after inserting the transcatheter device 100 (which will be described in further detail hereinafter). In aspects, the prosthetic heart valve leaflets 204 may be lacerated while concurrently inserting the transcatheter device 100 into the patient and/or may be lacerated with a lacerating feature of the transcatheter device 100. Alternatively, in some aspects, as shown in FIG. 4, the prosthetic heart valve leaflets 204 may not be lacerated wherein the heart valve may be replaced without lacerating the existing leaflets. For example, FIG. 4 shows an alternative embodiment of the preexisting prosthetic heart valve 200 of FIG. 2 where the prosthetic heart valve leaflets 204 have not been lacerated.

In further examples, the preexisting prosthetic heart valve 200 can comprise one or more skirts 213. In some aspects, the one or more skirts 213 can be coupled to the stent frame 202. In aspects, the one or more skirts 213 can comprise an inner skirt (as shown) or an outer skirt (not shown). The one or more skirts 213 can be coupled to any portion of the stent frame 202, such as for example, being coupled along the intermediate portion 205 and extending along the distal portion 203 to the distal end 212. The one or more skirts 213 can comprise any suitable skirt material, such as for example, a radiopaque fabric. It should be understood that the one or more skirts 213 described above are merely exemplary. Accordingly, the preexisting prosthetic heart valve 200 can comprise any other suitable skirt.

It should be understood that while the transcatheter device 100 will be referred to hereinafter as being utilized with the preexisting prosthetic heart valve 200, other suitable preexisting prosthetic heart valves known in the art may be utilized with the transcatheter device 100, heart valve prostheses (described in further detail later), and methods (described in further detail later). Furthermore, the transcatheter device 100 may also be utilized in a native heart valve (i.e., an aortic valve, a mitral valve, a tricuspid valve, and/or a pulmonic valve).

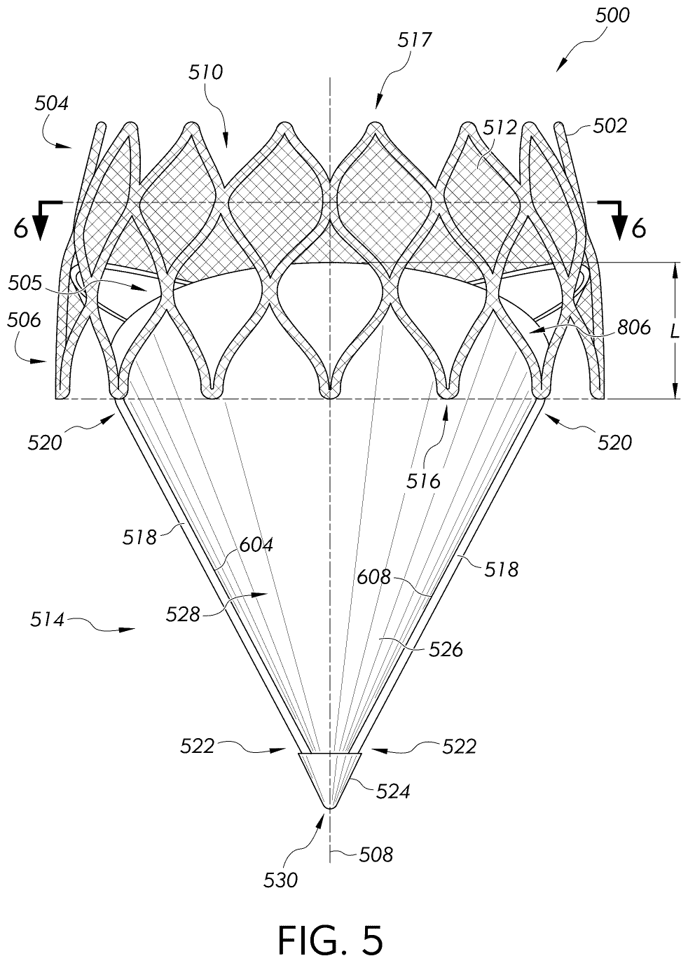

Turning to FIG. 5, an exemplary heart valve prosthesis 500 is shown. In aspects, the heart valve prosthesis 500 can comprise an expandable stent frame 502 configured to expand from a contracted orientation (shown in FIGS. 14 and 15) to an expanded orientation (as shown in FIG. 5). In aspects, the expandable stent frame 502 can comprise a proximal end portion 504 (outflow end) comprising a proximal end 517 and a distal end portion 506 (inflow end) comprising a distal end 516. The expandable stent frame 502 can extend from the proximal end portion 504 to the distal end portion 506 along an elongated axis 508 of the expandable stent frame 502. In some aspects, the expandable stent frame 502 can define a lumen 510 extending from the proximal end portion 504 to the distal end portion 506 along the elongated axis 508 of the expandable stent frame 502.

In some aspects, the expandable stent frame 502 can comprise a self-expandable stent frame. For example, the expandable stent frame 502 can comprise a self-expanding material. In some non-limiting examples, the self-expanding material can comprise nitinol. However, the expandable stent frame 502 should not be limited to nitinol, any other self-expanding materials known in the art may be utilized for the expandable stent frame 502.

In aspects, the expandable stent frame 502 can comprise a plurality of cell structures 505. In aspects, the plurality of cell structures 505 can comprise diamond-shaped cell structures. However, it should be understood that the expandable stent frame 502 is merely exemplary and any other suitable expandable stent frames may be utilized with the heart valve prosthesis 500 described herein. Furthermore, the plurality of cell structures 505 should not be limited to diamond-shaped cell structures and should not be limited to the number of cell structures and/or size of the cell structures shown in FIG. 5. Accordingly, more or less suitable cell structures with a variety of different shapes and sizes can be employed with the heart valve prosthesis 500.

In some aspects, the heart valve prosthesis 500 can further comprise an outer skirt 512 coupled to an outer surface of the expandable stent frame 502. The outer skirt 512 can extend along any length of the expandable stent frame 502, such as for example, the outer skirt 512 can extend along the entire length of the expandable stent frame 502 or half the length of the expandable stent frame 502. In other non-limiting examples, the outer skirt 512 can extend only along the proximal end portion 504 of the expandable stent frame 502 or the distal end portion 506 of the expandable stent frame 502. In other examples, the outer skirt 512 can extend along only a portion of the proximal portion the expandable stent frame 502 and/or only a portion of the distal end portion 506 of the expandable stent frame 502. In some examples, the outer skirt 512 can extend from at least a portion of the proximal end portion 504 of the expandable stent frame 502 to at least a portion of the distal end portion 506 of the expandable stent frame 502. The outer skirt 512 can be coupled to the expandable stent frame 502 by any suitable coupling means. For example, the outer skirt 512 can be coupled to the expandable stent frame 502 by stitching, such as for example, coupling the outer skirt 512 of the expandable stent frame 502 by a stitching pattern that extends from any portion of the proximal end portion 504 of the expandable stent frame 502 to any portion of the distal end portion 506 of the expandable stent frame 502. Any other suitable stitching pattern that extends along any portion of the proximal end portion 504 and/or the distal end portion 506 of the expandable stent frame 502 can be utilized. It should be understood that portions of the outer skirt 512 have been removed from FIG. 5 for clarity in order to show various features of the heart valve prosthesis 500, and thus is not meant to be limiting.

In some aspects (not shown), the heart valve prosthesis 500 can comprise an inner skirt coupled to an inner surface of the expandable stent frame 502 The inner skirt can extend along any length of the expandable stent frame 502, such as for example, the inner skirt can extend along the entire length of the expandable stent frame 502 or half the length of the expandable stent frame 502. In other non-limiting examples, the inner skirt can extend only along the proximal end portion 504 of the expandable stent frame 502 or the distal end portion 506 of the expandable stent frame 502. In other examples, the inner skirt can extend along only a portion of the proximal portion the expandable stent frame 502 and/or only a portion of the distal end portion 506 of the expandable stent frame 502. In some examples, the inner skirt can extend from at least a portion of the proximal end portion 504 of the expandable stent frame 502 to at least a portion of the distal end portion 506 of the expandable stent frame 502. The inner skirt can be coupled to the expandable stent frame 502 by any suitable coupling means. For example, the inner skirt can be coupled to the expandable stent frame 502 by stitching, such as for example, coupling the inner skirt of the expandable stent frame 502 by a stitching pattern that extends from any portion of the proximal end portion 504 of the expandable stent frame 502 to any portion of the distal end portion 506 of the expandable stent frame 502. Any other suitable stitching pattern that extends along any portion of the proximal end portion 504 and/or the distal end portion 506 of the expandable stent frame 502 can be utilized.

In some aspects, the outer skirt 512 and/or the inner skirt can comprise a radiopaque polymer. For example, a radiopaque polymer may be beneficial in facilitating a clinician in determining when the expandable stent frame 502 has been properly positioned within a patient. Any suitable polymer known in the art may be utilized. In other aspects, the outer skirt 512 and/or the inner skirt can comprise a fabric. For example, the fabric may comprise any suitable material such as, but not limited to, woven polyester such as polyethylene terephthalate, polytetrafluoroethylene (PTFE), or other biocompatible materials.

In some aspects, the heart valve prosthesis 500 can comprise a valve apparatus 514 that extends past the distal end 516 (inflow end) of the distal end portion 506 of the expandable stent frame 502. In some aspects, the valve apparatus 514 can comprise a plurality of commissure posts 518. For example, the valve apparatus 514 can comprise one commissure post, two commissure posts, three commissure posts, or any other suitable number of commissure posts. In some aspects, such as shown in FIG. 5, the valve apparatus 514 can comprise three commissure posts equidistantly spaced around the elongated axis 508. It should be understood that while FIG. 5 schematically illustrates three commissure posts, only two are shown because the third commissure post is positioned towards the back of the heart valve prosthesis 500 and is thus not visible. Additionally, while the plurality of commissure posts 518 are shown equidistantly spaced around the elongated axis 508, any other suitable spacing for any number of commissure posts of the plurality of commissure posts 518 may be utilized, such as for example, any number of commissure posts of the plurality of commissure posts 518 can be skewed around (e.g., not equidistantly spaced from one another) the elongated axis 508.

In further aspects, each commissure post (e.g., a first, a second, and a third commissure post) of the plurality of commissure posts 518 can comprise a first end portion 520 attached to the distal end portion 506 of the expandable stent frame 502. In aspects, each commissure post of the plurality of commissure posts 518 can be coupled to any portion of the distal end portion 506, such as for example, each commissure post can be coupled to the distal end 516 of the distal end portion 506 of the expandable stent frame 502 or along any of the plurality of cell structures 505 of the distal end portion 506. In alternative embodiments, each commissure post of the plurality of commissure posts 518 can be coupled along any portion of the expandable stent frame 502. For example, each commissure post of the plurality of commissure posts 518 can be coupled to the proximal end portion 504 and extend past the distal end 516 of the distal end portion 506 of the expandable stent frame 502. In further aspects, each commissure post of the plurality of commissure posts 518 can comprise a second end portion 522 attached to a distal tip 524 of the valve apparatus 514 at a distal end of the valve apparatus 514. In aspects, the plurality of commissure posts 518 can converge together in a distal direction of the elongated axis 508 towards the distal tip 524.

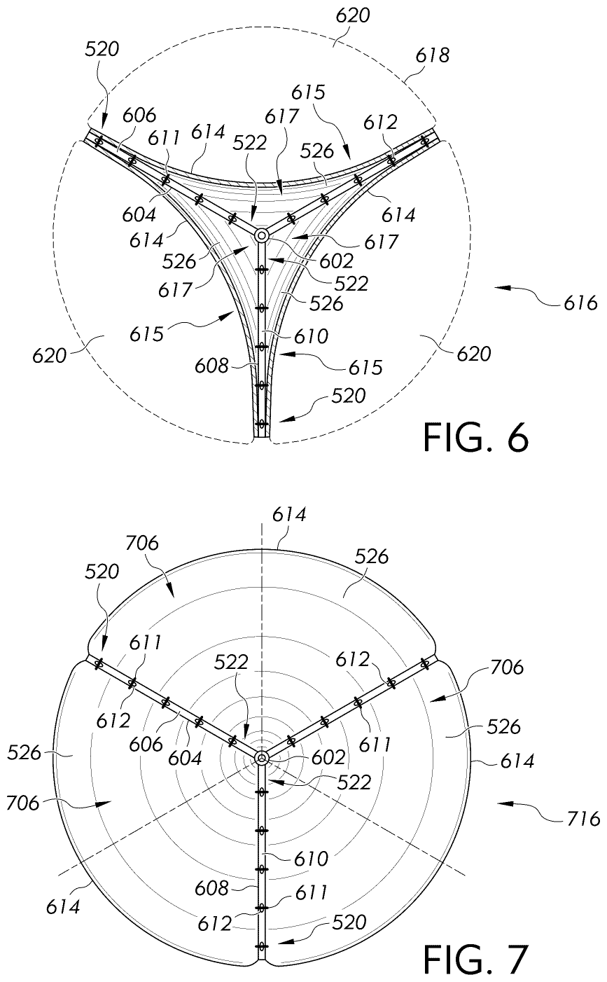

In some aspects, the second end portion 522 of each commissure post of the plurality of commissure posts 518 can be attached to the distal tip 524 by being received by the distal tip 524. In some examples, received by can mean received within, such as for example, the second end portion 522 can be received within a lumen 602 (see FIG. 6) of the distal tip 524. In some such examples, the second end portion 522 of each commissure post of the plurality of commissure posts 518 can be received within a single lumen of the distal tip 524 shared by each commissure post of the plurality of commissure posts 518. In some other such examples, the distal tip 524 can comprise a plurality of lumens that receive the second end portion 522 of each commissure post of the plurality of commissure posts 518. For example, the second end portion 522 of each commissure post of the plurality of commissure posts 518 can be received within its own corresponding lumen (not shown) of the plurality of lumens, such that the second end portion 522 of each commissure post of the plurality of commissure posts 518 is received within its respective unshared lumen within the distal tip 524 (e.g., a first lumen for a first commissure post, a second lumen for a second commissure post, etc.). In other examples, the distal tip 524 can comprise a plurality of lumens where the second end portion 522 of some (i.e., but not all) commissure posts of the plurality of commissure posts 518 share a lumen of the distal tip 524 while the second end portion 522 of some other different commissure posts share a different lumen of the distal tip 524. Any other suitable means of attaching the second end portion 522 of each commissure post of the plurality of commissure posts 518 to the distal tip 524 may be utilized, such as for example, attaching the second end portion 522 of each commissure post of the plurality of commissure posts 518 to an outer radial surface of the distal tip 524.

In some aspects, the distal tip 524 can further comprise a lumen 1402 extending through a distal apex 530 of the distal tip 524 (best shown in FIGS. 14 and 15). In some aspects, the lumen 1402 can be configured to receive an object. For example, the lumen 1402 can be configured to receive a guidewire therethrough to help navigate the heart valve prosthesis 500 and/or the transcatheter device 100 (explained in further detail later) through a patient's vasculature. In some embodiments, the distal tip 524 can be configured to comprise a radially expanded state (e.g., when an object is inserted therethrough) and a natural, contracted state (e.g., when an object is removed and/or no object is within the lumen 1402). For example, one or more portions (e.g., portions where the lumen 1402 is located) of the distal tip 524 can comprise a resilient material that has a tendency to return to its natural, contracted shape. Accordingly, the distal apex 530 where the lumen 1402 extends through can comprise a resilient material that maintains a contracted state when no forces are exerted on it, such that the lumen 1402 at the distal apex 530 is closed. Furthermore, in some examples, when an object is inserted through the distal tip 524 (e.g., such as inserting a guide wire therethrough) such that it reaches the distal apex 530, the force exerted on the distal apex 530 will cause the distal tip 524 to radially expand to the radially expanded state such that the lumen 1402 is open. In some such examples, when the object is removed (e.g., such as proximally retracting the guidewire from the lumen 1402), the distal tip 524 will return to its natural, contracted state (e.g., the lumen 1402 will close). In other examples, the distal tip 524 can comprise a flap or flaps that are configured to open and close when inserting or removing an object respectively (e.g., a valve, such as a duckbill valve or other types of valves). In other examples, the lumen 1402 can be configured to always be in an open state (e.g., does not radially contract). In yet another example, the lumen 1402 can be omitted from the distal apex 530 of the distal tip 524.

In further aspects (not shown), the distal tip 524 can comprise one or more apertures circumferentially positioned around the outer radial surface of the distal tip 524 and further extending through the outer radial surface of the distal tip 524 to an inner radial surface of the distal tip 524. In some examples, providing the one more apertures will help to minimize blood stagnation within the distal tip 524, such as for example, where the lumen 1402 is omitted from the distal tip 524, where the distal tip 524 is configured to maintain a natural, contracted state, or where the lumen 1402 is too small to properly prevent blood stagnation within the distal tip 524.

In some aspects, the distal tip 524 can be any suitable shape and/or size, such as for example, a rounded conical shape to be atraumatic, thereby minimizing/preventing damage while maneuvering the transcatheter device 100 through a patient's vasculature, as will become apparent hereinafter. In further aspects, the distal tip 524 can comprise any suitable material, such as for example, silicone, polyurethane, thermoplastic elastomers, radiopaque fillers (e.g., barium sulfate), and/or the like.

As further shown in FIG. 5, in aspects, the heart valve prosthesis 500 can comprise a plurality of leaflets 526 each coupled to a corresponding pair of the plurality of commissure posts 518. The terms “corresponding pair of the plurality of commissure posts” and “a first commissure post, a second commissure post, etc.,” are not meant to be limiting as to a number of commissure posts and/or which two commissure posts form the corresponding pair of commissure posts. As utilized herein, the term should be construed to refer to whichever two commissure posts (e.g., any two commissure posts) of the plurality of commissure posts 518 a particular leaflet of the plurality of leaflets 526 is being coupled to, and thus any pair of commissure posts may be referred to when discussing the corresponding pair of commissure posts. Accordingly, when examples are discussed herein with reference to a first commissure post, a second commissure post, and so on, this is not intended to limit the corresponding pair of commissure posts to only a first commissure post paired with only a second commissure post, etc. Rather, any commissure post may be construed as being a first commissure post or a second commissure post, and/or the like. For example, the corresponding pair of the plurality of commissure posts 518 can comprise a first commissure post and a second commissure post to form the corresponding pair of commissure posts, a second commissure post and a third commissure post can form the corresponding pair of commissure posts, a first commissure post and a third commissure post can form the corresponding pair of commissure posts, and/or the like.

In some embodiments, as illustrated, the plurality of leaflets 526 can comprise three leaflets (i.e., to emulate a tricuspid valve). In some such examples, the three leaflets can be circumferentially distributed around the elongated axis 508. In other examples more or less leaflets can be provided, such as for example, less than three leaflets (e.g., two leaflets to emulate a bicuspid valve). In some examples, the plurality of leaflets 526 can comprise the same number of leaflets as the heart valve (e.g., the preexisting prosthetic heart valve 200 or a native heart valve) that the heart valve prosthesis 500 is replacing, such that the plurality of leaflets 526 function similarly as the replaced valve.

With additional reference to FIGS. 6 and 7, in some aspects, the plurality of leaflets 526 each can comprise a first edge 604 attached along a first commissure post 606 of the corresponding pair of commissure posts from the first end portion 520 of the first commissure post 606 to the second end portion 522 of the first commissure post 606. In further aspects, the plurality of leaflets 526 can each comprise a second edge 608 attached along a second commissure post 610 of the corresponding pair of commissure posts from the first end portion 520 of the second commissure post 610 to the second end portion 522 of the second commissure post 610. The first edge 604 and the second edge 608 of each of the plurality of leaflets 526 can be attached to the corresponding pair of commissure posts any number of suitable ways. One non-limiting example of how the first edge 604 and the second edge 608 can be coupled to the corresponding pair of commissure posts is by stitching the first edge 604 and the second edge 608 to the corresponding pair of commissure posts by way of one or more threads 611. In some such non-limiting examples (as shown in FIGS. 6 and 7), the corresponding pair of commissure posts can comprise a plurality of apertures 612 (e.g., one or more apertures) that extend long a length of the commissure posts. The first edge 604 and the second edge 608 can then be stitched to the commissure posts by one or more threads 611 that extend through each of the plurality of apertures 612 (as shown). In some examples, the first edge 604 and the second edge 608 can be threaded around the corresponding pair of commissure posts and the plurality of apertures 612 can be removed. Any other suitable way of coupling the first edge 604 and the second edge 608 of each of the plurality of leaflets 526 to the corresponding pair of commissure posts may be utilized.

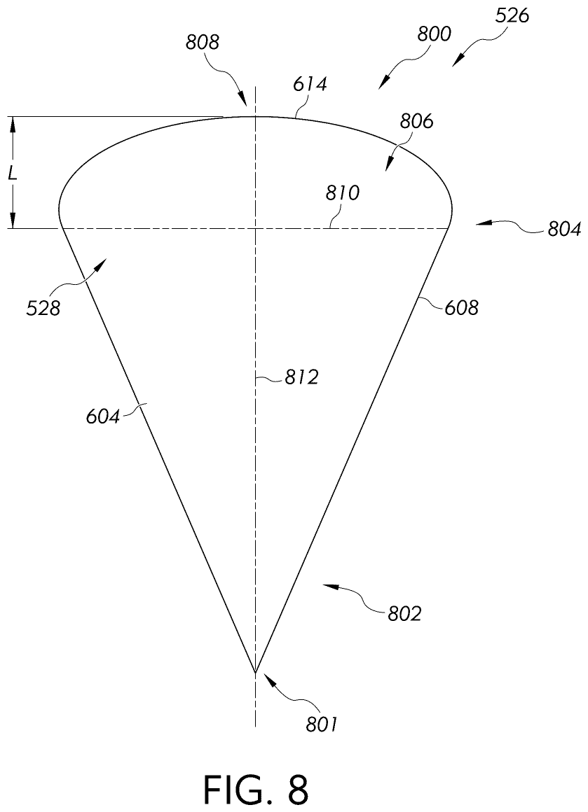

In some aspects, the plurality of leaflets 526 can each comprise an third edge 614 configured to alternate between an inwardly bent configuration 616 (shown in FIG. 6) and an outwardly bent configuration 716 (shown in FIG. 7), described in further detail with reference to FIG. 8. For example, as shown in FIG. 6, the third edge 614 is bent radially inward relative to the elongated axis 508 when in the inwardly bent configuration 616. In some aspects, the plurality of leaflets 526 can each comprise a convex surface 617 facing the elongated axis 508 in the inwardly bent configuration 616. The convex surface 617 is schematically illustrated in FIG. 6 as an arcuate surface that extends into the page along the third edge 614 and further extends between the first edge 604 and the second edge 608 of each leaflet of the plurality of leaflets 526. In aspects, the plurality of leaflets 526 can each comprise a corresponding concave surface 615 (e.g., generally shown as the surface that extends into the page) that faces away from the elongated axis 508 in the inwardly bent configuration 616. The dashed line circle 618, shown in FIG. 6, generally illustrates where the third edge 614 would be when each leaflet of the plurality of leaflets 526 are in the outwardly bent configuration 716 as shown in FIG. 7. In some aspects, the inwardly bent configuration 616 can comprise an opening 620 defined between the expandable stent frame 502 and each of the plurality of leaflets 526. In order to demonstrate the inwardly bent configuration 616, a non-limiting example of the functionality of the heart valve prosthesis 500 will be described. As a non-limiting example to demonstrate the inwardly bent configuration 616, in an embodiment where the heart valve prosthesis 500 is implanted within a native heart valve and/or an implanted prosthetic heart valve (e.g., the preexisting prosthetic heart valve 200), when the cardiac cycle is in systole (i.e., the period in which the ventricle contracts to force blood into the arteries), each leaflet of the plurality of leaflets 526 can be in the inwardly bent configuration 616, in which each leaflet of the plurality of leaflets defines the opening 620 to permit blood to flow around each of the plurality of leaflets 526. The opening 620, in which blood may flow around each of the plurality of leaflets 526, can be schematically illustrated between the dashed line circle 618 and the third edge 614 of each of the plurality of leaflets 526 while in the inwardly bent configuration 616.

As shown in FIG. 7, the third edge 614 can be bent radially outward relative to the elongated axis 508 when in the outwardly bent configuration 716. In some aspects, the plurality of leaflets 526 can each comprise a convex surface 528 (see FIG. 5) facing away from the elongated axis 508 in the outwardly bent configuration 716. In aspects, the plurality of leaflets 526 can each comprise a corresponding (e.g., corresponding to the convex surface 528 of each of the plurality of leaflets 526) concave surface 706 facing the elongated axis 508 in the outwardly bent configuration 716. The convex surface 528 is schematically illustrated in FIG. 7 as an arcuate surface that extends into the page along the third edge 614 and further extends between the first edge 604 and the second edge 608 of each leaflet of the plurality of leaflets 526.

It should be understood that the convex surface 617 (shown in FIG. 6) and the concave surface 706 (shown in FIG. 7) are labeled herein with different reference numerals for clarity to distinguish between the inwardly bent configuration 616 and the outwardly bent configuration 716. However, the convex surface 617 and the concave surface 706 represent the same surface of each leaflet of the plurality of leaflets 526 but in the different configurations (e.g., the inwardly bent configuration 616 and, the outwardly bent configuration 716). Furthermore, the concave surface 615 (shown in FIG. 6) and the convex surface 528 (shown in FIG. 5) are labeled herein with different reference numerals for clarity to distinguish between the inwardly bent configuration 616 and the outwardly bent configuration 716. However, the concave surface 615 and the convex surface 528 represent the same surface of each leaflet of the plurality of leaflets 526 but in the different configurations (e.g., the inwardly bent configuration 616 and the outwardly bent configuration 716). Thus, as should be clear herein, in some configurations (e.g., the inwardly bent configuration 616 and the outwardly bent configuration 716) a surface may be concave and then in another configuration the same surface may turn convex, or conversely, a convex surface may become concave.

In some aspects, the plurality of leaflets 526 can be configured to form a conical configuration. For example, as illustrated in FIGS. 5 and 7, when the plurality of leaflets 526 each comprise a convex surface 528 facing away from the elongated axis 508, each leaflet of the plurality of leaflets together can form to the conical configuration. For example, by coupling the first edge 604 and the second edge 608 of each of the plurality of leaflets 526 to a corresponding pair of commissure posts, each leaflet can work in tandem to form the conical configuration. As a non-limiting example to demonstrate the conical configuration, in an embodiment where the heart valve prosthesis 500 is implanted within a native heart valve and/or the preexisting prosthetic heart valve 200 (described above), when the cardiac cycle is in diastole (i.e., the period in which the ventricle fills with blood), each leaflet of the plurality of leaflets 526 can be in the outwardly bent configuration 716 to prevent blood from flowing around each of the plurality of leaflets 526. Accordingly, when each leaflet of the plurality of leaflets 625 are in the outwardly bent configuration 716, the opening 620 between the dashed line circle 618 and the third edge 614 is removed, thereby preventing blood from flowing therethrough.

Turning to FIG. 8, a two-dimensional schematic side view of one acceptable embodiment of each leaflet 800 of the plurality of leaflets 526 is shown. For the purposes of clarity, only a single leaflet is shown in which the leaflet 800 is free from any surrounding structure (e.g., not coupled to the pair of corresponding commissure posts). It should be understood that each leaflet of the plurality of leaflets may comprise the same or different leaflets as the leaflet 800 described hereinafter. It should further be understood that the leaflet 800 can comprise any features previously described for each of the plurality of leaflets 526 and thus will be referenced hereinafter with further reference to FIG. 8.

In some aspect, each leaflet 800 of the plurality of leaflets 526 can comprise a distal peak 801. In aspects, the first edge 604 can extend proximally in a first direction from the distal peak 801 and the second edge 608 can extend proximally in a second direction from the distal peak 801. In aspects, the third edge 614 can connect the first edge 604 and the second edge 608. In further aspects, the plurality of leaflets 526 can each comprise a distal portion 802 (inflow portion) and a proximal portion 804 (outflow portion). In aspects, one or more portions of each leaflet 800 of the plurality of leaflets 526 can extend proximally past the distal end 516 of the expandable stent frame 502 towards the proximal end portion 504 of the expandable stent frame 502. In one non-limiting example, as shown, a portion 806 of the proximal portion 804 of each leaflet 800 of the plurality of leaflets 526 can extend proximally past the distal end 516 of the expandable stent frame 502 towards the proximal end portion 504 of the expandable stent frame 502 (see FIG. 5). In aspects, the portion 806 of the proximal portion 804 of each leaflet 800 can comprise a rounded portion that can extend proximally past the distal end 516 of the expandable stent frame 502. In some such aspects, the rounded portion can be defined by an arcuate edge of the third edge 614, as shown. It should be understood that while the leaflet 800 shows a rounded portion extending past the distal portion other shapes can also be suitable. For example, the portion 806 that extends past the distal end 516 of the expandable stent frame 502 can comprise a parallelogram shape or a trapezoidal shape defined by the third edge 614. In other aspects, the leaflet 800 can comprise a pair of vertical or angled edges (not illustrated) defined by the third edge 614 that extend from an upper leaflet portion 810 (indicated by a dashed line). In aspects, the upper leaflet portion 810 can be defined as a horizontal line taken across the leaflet 800 at a location where the third edge 614 connects the first edge 604 and the second edge 608.

In aspects, the portion 806 of the proximal portion 804 of each leaflet 800 that can proximally extend past the distal end 516 of the expandable stent frame 502 can be defined by a length L. The length L can be any number of suitable lengths. In some embodiments, the length L can be defined by a distance from a proximal apex 808 (indicated generally by an arrow) of the third edge 614 to the upper leaflet portion 810. It should be understood from above that the portion 806 of the proximal portion 804 of each leaflet 800 that can extend proximally past the distal end 516 of the expandable stent frame 502 can comprise a variety of other shapes defined by the third edge 614 (e.g., vertical or angled edges), and thus while the length L has been described above as being defined in terms of the proximal apex 808 of the third edge 614, the length L may change depending on the shape of the portion 806. For example, if the portion 806 of the proximal portion 804 of each leaflet 800 comprises a parallelogram defined by the third edge 614, the length L can be defined by the proximal most point(s) of the parallelogram to the upper leaflet portion 810 of the first edge 604 and the second edge 608. In other embodiments not shown, the length L can extend from any other portion of the leaflet 800, such as for example, from the proximal apex 808 to any portion along the first edge 604 and the second edge 608. By allowing the portion 806 of the proximal portion 804 of each leaflet 800 to proximally extend past the distal end 516 of the expandable stent frame 502, the heart valve prosthesis 500 will be able to greater resist regurgitation (i.e., leakage of blood backwards through the heart valve prosthesis 500)

As previously described above in FIGS. 6 and 7, each leaflet 800 of the plurality of leaflets 526 can be configured to alternate between the inwardly bent configuration 616 and the outwardly bent configuration 716. In aspects, as shown in FIG. 8, each leaflet can comprise an extended axis 812 that generally bisects each leaflet 800 from the proximal apex 808 to the distal peak 801 (indicated generally by an arrow). The extended axis 812 is illustrated schematically to further explain the functionality of both the inwardly bent configuration 616 and the outwardly bent configuration 716. For example, during both the inwardly bent configuration 616 and the outwardly bent configuration 716 each leaflet 800 can bend along the extended axis 812. For example, to achieve the inwardly bent configuration 616 (e.g., during systole) each leaflet 800 can bend radially inward (in a direction into the page as viewed from FIG. 8) along the extended axis 812. Accordingly, the plurality of leaflets 526 each can comprise the convex surface 617 that faces the elongated axis 508 (this would be the surface that is not shown in FIG. 8). Alternatively, to achieve the outwardly bent configuration 716 (e.g., during diastole) each leaflet 800 can bend radially outward (in a direction away from the page as viewed from FIG. 8) along the extended axis 812. Accordingly, the plurality of leaflets 526 each can comprise the convex surface 528 that faces away from the elongated axis 508 (this would be the surface that is shown in FIG. 8). It should be understood that because FIG. 8 represents a two-dimensional view of the leaflet 800, a convex surface is not schematically visible in FIG. 8.

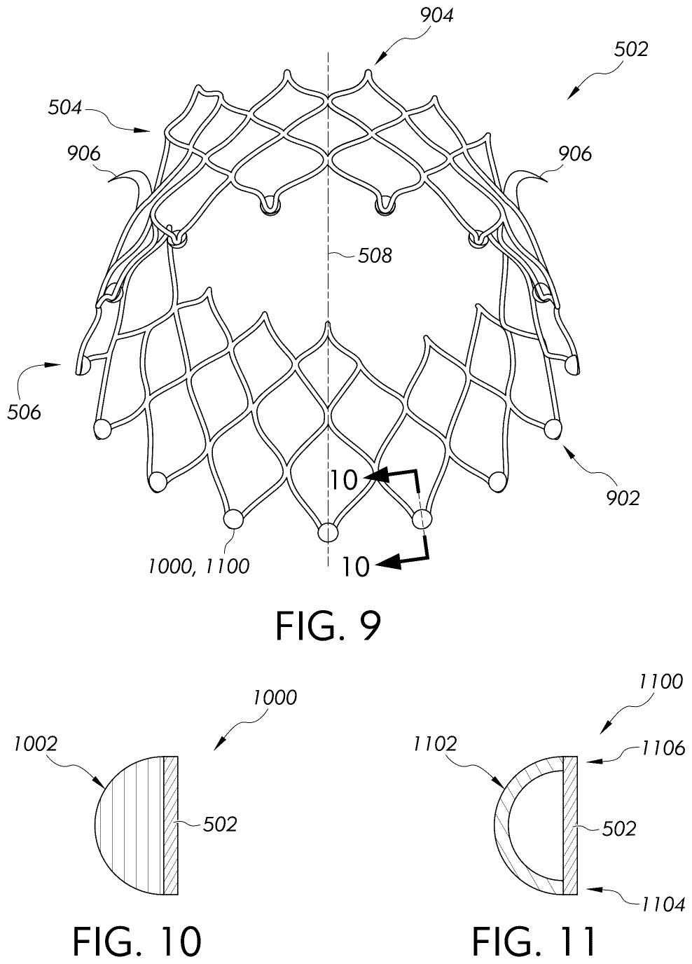

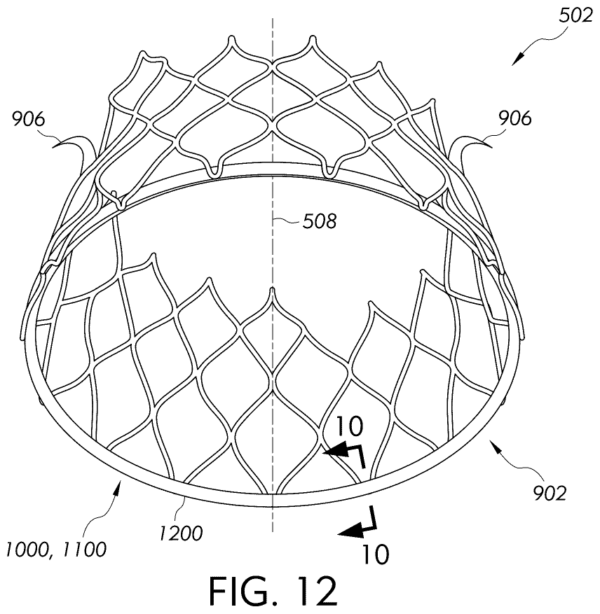

Turning to FIGS. 9-12, the heart valve prosthesis 500 can further comprise a hemostatic bumper 1000, 1100 coupled to the expandable stent frame 502. More specifically, FIGS. 9 and 12 show a bottom perspective view the expandable stent frame 502 of the heart valve prosthesis 500 with the hemostatic bumper 1000, 1100 coupled to the expandable stent frame 502 while FIGS. 10 and 11 show a schematic cross sectional view of particular embodiments of the hemostatic bumper 1000, 1100 utilized with the expandable stent frame 502 shown in either one of FIGS. 9 and 12.

FIG. 10 shows the hemostatic bumper 1000. In aspects, the hemostatic bumper 1000 can comprise a solid hemostatic bumper. The solid hemostatic bumper can comprise any suitable material, such as for example, but not limited to, alloys/metals, plastics, polymers and/or the like. Alternatively, the hemostatic bumper 1000 can comprise a hollow shell formed from any suitable material, such as those listed above for the solid hemostatic bumper. In further aspects, as shown, the hemostatic bumper 1000 can comprise an outer spherical surface 1002 that radially extends in a direction towards the elongated axis 508. In some embodiments not shown, the hemostatic bumper 1000 can comprise a variety of other suitable shapes and sizes. For example, more generally the hemostatic bumper can comprise a protrusion. In some such examples, the protrusion can comprise a hollow or solid protrusion that radially extends in a direction towards the elongated axis 508. The protrusion can comprise a square protrusion, a rectangular protrusion, a triangular protrusion, a curved protrusion, and/or the like.

FIG. 11, shows an alternative embodiment of the hemostatic bumper 1100. In aspects, the hemostatic bumper 1100 can comprise a strand of material coupled to the expandable stent frame 502. For example, the strand of material can comprise an intermediate portion 1102 extending between a first end portion 1104 coupled to the expandable stent frame 502 and a second end portion 1106 coupled to the expandable stent frame 502. The first end portion 1104 and the second end portion 1106 can be coupled to the same portion or different portions of the expandable stent frame 502, such that the intermediate portion 1102 of the strand of material bends radially outward towards the elongated axis 508 to form a substantially convex outer surface. In other examples, not shown, the strand of material can radially extend towards the elongated axis 508 but comprise various other shapes, such as for example, rectangular strands of material, polygonal strands of material, and/or the like. The strand of material can comprise any suitable material, such as for example, those listed above for the hemostatic bumper 1000.

As shown in FIG. 9, in aspects the hemostatic bumper 1000, 1100 can comprise a plurality of hemostatic bumpers circumferentially disposed around the elongated axis 508. In some aspects, the plurality of hemostatic bumpers can comprise a combination (e.g., any combination) of the hemostatic bumpers 1000, 1100 described above. For example, the plurality of hemostatic bumpers can comprise an alternating pattern around the elongated axis 508 of the hemostatic bumper 1000 and the hemostatic bumper 1100. Any other suitable pattern or combination for the plurality of hemostatic bumpers can be utilized.

In aspects, each hemostatic bumper 1000, 1100 of the plurality of hemostatic bumpers can be disposed on at least a portion of a distal crown of a plurality of distal crowns 902 (inflow crowns) of the distal end portion 506 of the expandable stent frame 502. In some examples, the hemostatic bumper 1000, 1100 or combination thereof can be provided on each distal crown of the plurality of distal crowns 902, as shown in FIG. 9. In other examples, only some distal crowns of the plurality of distal crowns 902 can comprise a hemostatic bumper 1000, 1100. In yet another example, only one distal crown of the plurality of distal crowns 902 can be provided with the hemostatic bumper 1000, 1100. In other examples not shown, the hemostatic bumper 1000, 1100 can be provided on other portions of the expandable stent frame 502, such as for example, one or more proximal crowns of a plurality of proximal crowns 904 of the proximal end portion 504 of the expandable stent frame 502. Any other suitable positions along an interior surface or exterior surface of the expandable stent frame 502 can be provided with the hemostatic bumper 1000, 1100.

In will be appreciated that by varying the dimensions (e.g., diameter, length, width, etc.) and/or shape (circular protrusions, square protrusions, convex strands of material, polygonal strands of material etc.) of the hemostatic bumper 1000, 1100, hemostatic forces exerted on the heart valve prosthesis 500 as a result of blood flowing through the heart valve prosthesis 500 can be removed/minimized at portions where the hemostatic bumper is positioned on the expandable stent frame 502.

Turning to FIG. 12, in some aspects, the hemostatic bumper 1000, 1100 can comprise a continuous band 1200 (e.g., disk/ring shaped) disposed around at least a portion of the elongated axis 508. In some aspects, the hemostatic bumper 1000, 1100 can comprise a continuous band 1200 (e.g., disk/ring shaped) disposed around the entire elongated axis 508 (e.g., can be disposed 360 degrees around the elongated axis). For example, the continuous band 1200 can be disposed around the elongated axis 508 (e.g., less than or equal to 360 degrees around the elongated axis 508) such that the continuous band 1200 continuously circumscribes (e.g., an entire circumference of the expandable stent frame 502, half the circumference of the expandable stent frame 502, etc.) the interior surface of the expandable stent frame 502 along at least a portion of a length of the expandable stent frame (e.g., continuously circumscribes the interior surface along the distal end portion 506 of the expandable stent frame 502). In some examples, as illustrated in FIG. 12, the continuous band 1200 can continuously circumscribe at least some or all of the plurality of distal crowns 902 along the interior surface of the expandable stent frame 502. In some aspects, the continuous band 1200 can be formed any number of suitable ways, such as for example, by elongating (e.g., through extrusion or any other manufacturing technique) the hemostatic bumper 1000, 1100. Accordingly, as should be apparent from above, taking a cross-section along any portion of the continuous band 1200 will result in the same or similar cross section as shown in either one of FIG. 10 or 11. For example, taking a cross-section along line 10-10 of FIG. 12 will result in a cross-section as described previously with reference to either one of FIG. 10 or 11. In this way, the continuous band 1200 can comprise a protrusion (e.g., as described above with reference to FIG. 10), such as for example, a hollow or solid protrusion (e.g., as described above) that radially extends in a direction towards the elongated axis 508. Alternatively, in some embodiments, when a cross section is taken along line 10-10 of FIG. 12, the continuous band 1200 can comprise a strand of material (e.g., as described above with reference to FIG. 11) coupled to the expandable stent frame 502. For example, the strand of material can comprise an intermediate portion 1102 extending between a first end portion 1104 coupled to the expandable stent frame 502 and a second end portion 1106 coupled to the expandable stent frame 502 (e.g., as described above). The first end portion 1104 and the second end portion 1106 can be coupled to the same portion or different portions of the expandable stent frame 502, such that the intermediate portion 1102 of the strand of material bends radially outward towards the elongated axis 508 to form a substantially convex outer surface.

As further shown in FIGS. 9 and 12, in some aspects, the expandable stent frame 502 can further comprise a plurality of protrusions 906 circumferentially spaced around the elongated axis 508. The plurality of protrusions 906 can comprise any number of suitable shapes/and or sizes. For example, in aspects, each protrusion of the plurality of protrusions 906 can comprise hooks, bumps, spikes, anchors, barbs, tines, teeth, and/or the like. In aspects, each protrusion of the plurality of protrusions 906 can be the same protrusion (e.g., hooks, bumps, spikes, etc.), each protrusion of the plurality of protrusions 906 can be a different protrusion, or some protrusion of the plurality of protrusions 906 can be the same protrusions while other protrusions of the plurality of protrusions 906 can be different protrusions (e.g., the same different protrusion or other different protrusions). Any other suitable combination for the plurality of protrusions 906 can be utilized. The plurality of protrusions 906 can comprise any number of protrusions, such as for example, one protrusion, two protrusions, three protrusions, four protrusions, and so on.

In some aspects, the plurality of protrusions 906 can be circumferentially and equidistantly spaced around the elongated axis 508. In other aspects, the plurality of protrusions 906 can be circumferentially skewed (i.e., not equidistantly spaced) around the elongated axis 508. In aspects, each protrusion of the plurality of protrusions 906 can extend from the expandable stent frame 502 in a direction away from the elongated axis 508. In further aspects, the plurality of protrusions 906 can further be provided along any or all lengths of the expandable stent frame 502. For example, the plurality of protrusions 906 can be positioned on either one of the proximal end portion 504 or the distal end portion 506. In other examples, the plurality of protrusions 906 can be positioned on both the proximal end portion 504 and the distal end portion 506. Any other suitable length and/or lengths along the expandable stent frame 502 for positioning the plurality of protrusions 906 can be utilized. It will become more apparent hereinafter that the plurality of protrusions 906 will be beneficial in helping the heart valve prosthesis 500 index with a heart valve, such as for example a native heart valve (e.g., a tricuspid valve, a pulmonary valve, a mitral valve, and/or an aortic valve) and/or the preexisting prosthetic heart valve 200 described above.

It should be understood that the FIGS. 9-12 are illustrated without showing the other components (e.g., the valve apparatus 514, the outer skirt 512, etc.) for clarity. Accordingly, it should be clear that the plurality of protrusions 906 are meant to work in combination with any of the previously described (and hereinafter described) components and/or embodiments of the transcatheter device 100 and/or the heart valve prosthesis 500 unless otherwise specifically states.

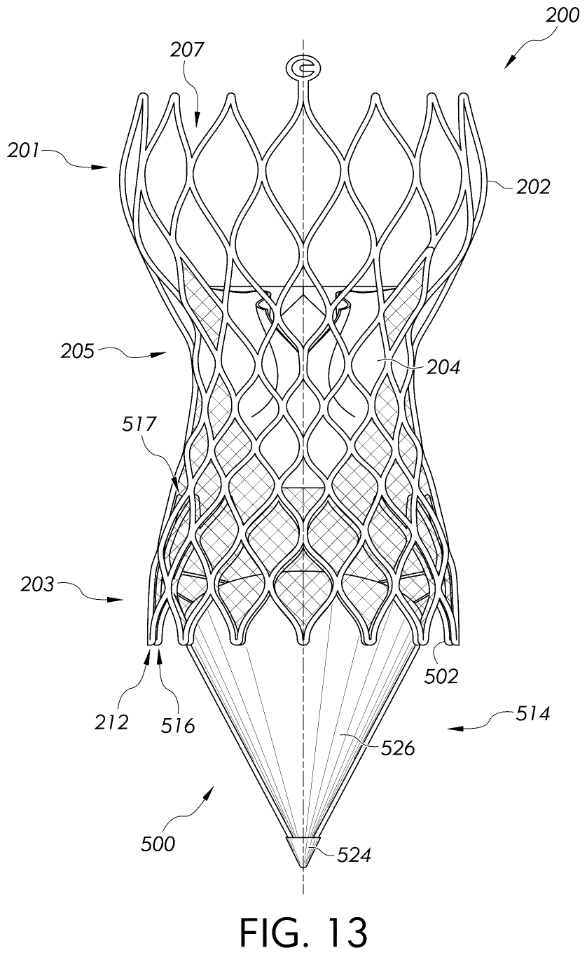

Turning to FIG. 13, the heart valve prosthesis 500 is illustrated where it has been inserted into the lumen 207 of the preexisting prosthetic heart valve 200. In aspects, as shown in FIG. 13, the expandable stent frame 502 of the heart valve prosthesis 500 is configured to index with the stent frame 202 of the preexisting prosthetic heart valve 200. In some examples, “index” can mean contacting, engaging, holding, interlocking, interconnecting, joining, mating, coupling, and/or the like. For example, the proximal end portion 504 and the distal end portion 506 can index with an inner region of the distal portion 203 of the stent frame 202 of the preexisting prosthetic heart valve 200.

In some examples, the proximal end portion 504 and the distal end portion 506 of the expandable stent frame 502 will index by way of a radial expansion force while in the expanded orientation. For instance, as shown in FIG. 13, the proximal end portion 504 and the distal end portion 506 of the expandable stent frame 502 is expanded such that the proximal end portion 504 and the distal end portion 506 of the expandable stent frame 502 is biased against the inner region of the distal portion 203 of the stent frame 202. In this way, the expandable stent frame 502 will resist lateral and axial movement, when in the expanded orientation. In some aspects, not specifically shown in FIG. 13, the plurality of protrusions 906 (e.g., spikes, bumps, etc.) will help the heart valve prosthesis 500 index with the stent frame 202. For example, the plurality of protrusions 906 can index (e.g., interlock, engage, etc.) with the stent frame 202 (e.g., the distal portion 203 of the stent frame 202) to further resist movement (e.g., the expandable stent frame 502 will further resist lateral and axial movement when in the expanded orientation).

As further shown in FIG. 13, when the heart valve prosthesis 500 has indexed with the preexisting prosthetic heart valve 200, the valve apparatus 514 can extend distally past the distal end 212 of the preexisting prosthetic heart valve (described in greater detail later). It should be further understood from above that while the heart valve prosthesis 500 has been described with reference to the preexisting prosthetic heart valve 200, the heart valve prosthesis 500 can be utilized in combination with a native heart valve and can function similarly to that described above with reference to the preexisting prosthetic heart valve 200.

FIGS. 14-15 will now be utilized to discuss various embodiments of the transcatheter device 100. More specifically, FIGS. 14-15 will be utilized to discuss various embodiment of the distal end portion 1400, 1500 of the transcatheter device 100. Accordingly, the various embodiments of the distal end portion 1400, 1500 of the transcatheter device 100 will be described hereinafter with initial reference to FIGS. 2-13. As shown, the transcatheter device 100 can comprise the outer shaft 102, an inner shaft 1404, the expandable stent frame 502, and the valve apparatus 514.

In aspects, the inner shaft 1404 can extend within the outer shaft 102 and through a distal end 1408 of the outer shaft 102. In some aspects, the inner shaft 1404 and the outer shaft 102 can be configured to translate relative to one another. The term “translate” as used herein, can mean that the inner shaft 1404 and/or the outer shaft 102 can move, slide, shift, and/or the like, relative to one another. In some examples, the inner shaft 1404 and/or the outer shaft 102 can be configured to proximally and/or distally translate relative to one another. In further examples, the inner shaft 1404 and/or the outer shaft 102 can also be configured to rotate (e.g., 360 degrees) relative to one another.

In aspects, the inner shaft 1404 can comprise an inner lumen 1406 configured to receive an object. For example, the inner lumen 1406 can be configured to receive a guide wire. In further aspects, the inner lumen 1406 of the inner shaft 1404 can align with the lumen 1402 of the distal tip 524 of the valve apparatus 514 along the elongated axis 508 of the expandable stent frame 502 such that when object passes through the inner lumen 1406 of the inner shaft 1404 it can also pass through the lumen 1402 of the distal tip 524. In further aspects, a distal segment 1410 of the inner shaft 1404 distally extends outside of the distal end 1408 of the outer shaft 102 and through the lumen 510 of the expandable stent frame 502.

Turning now specifically to the distal end portion 1400 of the transcatheter device 100 illustrated in FIG. 14, an embodiment is shown where the expandable stent frame 502 can comprise a self-expandable stent frame (such as described previously). In some aspects, as shown the expandable stent frame 502 can be disposed around the distal segment 1410 of the inner shaft 1404. In some aspects, distal segment 1410 of the inner shaft 1404 can comprise a crown capture mechanism to prevent movement (e.g., axial/lateral shifting) of the expandable stent frame, such as for example, one or more apertures, grooves, indents, cutouts, pockets and/or the like disposed on one or more portions of the distal segment 1410 of the inner shaft 1404. In some such aspects, the one or more apertures, grooves, indents, cutouts, pockets and/or the like can be configured to receive one or more of the distal crowns 902 and/or one or more of the proximal crowns 904. In some further example, a portion of the inner shaft 1404 can comprise one or more apertures, grooves, indents, cutouts, pockets and/or the like to capture the distal crowns 902, while another portion of the inner shaft 1404 can comprise one or more apertures to capture the proximal crowns 904. In some further examples, the distal crowns 902 and/or the proximal crowns 904 can comprise one or more paddles that are received within the crown capture mechanism.

In some aspects, the transcatheter device 100 can further comprise a capsule 1412 attached to the distal end 1408 of the outer shaft 102. In some aspects, the expandable stent frame 502 is configured to be positioned within the capsule 1412 in the contracted orientation (as shown) while the valve apparatus 514 can distally extend from a distal end 1414 of the capsule 1412. In some aspects, the capsule 1412 can be attached to the distal end 1408 of the outer shaft 102 any suitable way, such as for example, the capsule 1412 can comprise a coupling element that is received by the distal end 1408 of the outer shaft 102. In other examples, the outer shaft 102 and the capsule 1412 can be formed as a monolithic structure. In further aspects, the capsule 1412 can be configured to translate (e.g., slide, shift, etc.) simultaneously with the outer shaft 102 such that it can translate relative to the inner shaft 1404. As should be apparent from FIG. 14, in some examples, due to the atraumatic shape of the valve apparatus 514, the valve apparatus 514 can define a portion of the transcatheter device 100, as shown. In this way, the valve apparatus 514 can be directly exposed to a patient's vasculature without damaging it. Furthermore, by extending the valve apparatus 514 past the distal end 1414 of the capsule 1412 to define a portion of the transcatheter device 100, the number of components of the transcatheter device 100 can be reduced, such as for example, not requiring an atraumatic tip to be separately coupled to a distal end of the transcatheter device 100 (e.g., the distal tip 524 acts as, and replaces, a tapered atraumatic tip of a typical delivery system). In some examples, the valve apparatus 514 will be able to further help a clinician to safely navigate the transcatheter device 100 through a patient's vasculature.

Now viewing specifically the distal end portion 1500 of the transcatheter device 100 illustrated in FIG. 15, an embodiment is shown where the expandable stent frame 502 is not a self-expandable stent frame. In some aspects, the transcatheter device 100 can comprise an expansion device 1502 positioned within the lumen 510 of the expandable stent frame 502 and extends along the elongated axis 508 of the expandable stent frame 502. In some such aspects, the expandable stent frame 502 can be disposed (e.g., crimped) around the expansion device 1502. In some further aspects, the expansion device 1502 can comprise a balloon. For example, the expansion device 1502 can comprise a balloon configured to receive saline, a contrast solution, or compressed air that may be delivered to the balloon to expand the expandable stent frame 502 from the contracted orientation to the expanded orientation. In some aspects, as shown in FIG. 15, when the expandable stent frame 502 is in the contracted orientation (e.g., disposed around the expansion device 1502), the proximal end 517 of the expandable stent frame 502 can abut the distal end 1408 of the outer shaft 102. In some such aspects, a substantially flush radial transition can be defined between an outer radial surface 1504 of the expandable stent frame 502 and an outer radial surface 1506 of the outer shaft 102. As a result, the expandable stent frame 502 can comprise a portion of the transcatheter device 100 as shown. In this way, the capsule 1412, as described above, can be removed from the distal end 1408 of the outer shaft 102. Accordingly, when the transcatheter device 100 is maneuvered (e.g., by a clinician) through the vasculature of a patient the expandable stent frame 502 and the valve apparatus 514 will be directly exposed to the surrounding vasculature of the patient. Similarly, as described above with reference to FIG. 14, in some examples, due to the atraumatic shape of the valve apparatus 514, the valve apparatus 514 can define a portion of the transcatheter device 100, as shown. In this way, the valve apparatus 514 (and the expandable stent frame 502) can be directly exposed to a patient's vasculature without damaging it. Furthermore, with the valve apparatus 514 and the expandable stent frame 502 defining a portion of the transcatheter device 100, the number of components of the transcatheter device 100 can be reduced, such as for example, not requiring an atraumatic tip or capsule to be separately coupled to a distal end of the transcatheter device 100. In some examples, the valve apparatus 514 will be able to further help a clinician to safely navigate the transcatheter device 100 through a patient's vasculature.

It should be noted that while FIG. 15 shows an embodiment of the distal end portion 1500 where the capsule 1412 has been removed from the transcatheter device 100, in some embodiments the capsule 1412 can still be provided. For example, the capsule 1412 can be attached to the distal end 1408 of the outer shaft 102 as described with reference to FIG. 14. In such an embodiment, the capsule 1412 can then be translated (e.g., proximally translated) to expose the expandable stent frame 502 prior to expanding the expansion device 1502. In some examples, it may be beneficial to provide the capsule 1412 with the distal end portion 1500 when the outer radial surface 1504 of expandable stent frame 502 comprises surface discontinuities that may damage a patient's vasculature, such as for example, when the expandable stent frame 502 is provided with the plurality of protrusions 906, as described above with reference to FIGS. 9 and 12.

The term “expanded orientation” should be construed to mean that the expandable stent frame 502 cannot expand any further with respect to the location at which the expandable stent frame 502 has been deployed (i.e., fully released from the transcatheter device 100). For example, when the expandable stent frame 502 is discussed with reference to the preexisting prosthetic heart valve 200, the term “expanded orientation” will refer to the expandable stent frame 502 being fully released from the transcatheter device 100 whereby the expandable stent frame 502 has engaged the preexisting prosthetic heart valve 200 such that the expandable stent frame 502 can no longer expand. Accordingly, the term “expanded orientation” can mean that the expandable stent frame 502 cannot radially expand any further with respect to the location at which it has been deployed, but may still be capable of further radial expansion when the expandable stent frame 502 is free from surrounding structure. In some cases, the term “expanded orientation” may refer to the expandable stent frame 502 not being capable of any further radial expansion regardless of the location at which the expandable stent frame 502 is deployed (i.e., the expandable stent frame 502 cannot radially expand any further, even when free from surrounding structure).

FIG. 16 illustrates an exploded view of an embodiment of the transcatheter device 100 illustrating the handle device 1600 comprising one or more actuators 1602. In aspects, the one or more actuators 1602 can be configured to actuate the capsule 1412 (e.g., by a sliding movement of the actuator). For example, the one or more actuators 1602 can proximally advance and/or distally retract the capsule 1412. In some aspects, the one or more actuators 1602 can be configured to rotate (e.g., 360 degree) the outer shaft 102 and/or the capsule 1412. In some examples, the one or more actuators 1602 can proximally and/or distally retract the capsule 1412 by way of the outer shaft 102 coupled to the capsule 1412. In some aspects, as shown, the one or more actuators 1602 can be configured to actuate the inner shaft 1404. For example, the one or more actuators 1602 can proximally advance and/or distally retract the inner shaft 1404. In some aspects, the one or more actuators 1602 can be configured to rotate (e.g., 360 degree) the inner shaft 1404. In some embodiments the inner shaft 1404 can comprise a shaft assembly. For example, one or more shafts can be coupled together to define the inner shaft 1404.

In some aspects, the handle device 1600 can be configured to actuate components of the transcatheter device 100 by any suitable actuating means. For example, the handle device 1600 can actuate components of the transcatheter device 100 by sliding movements (e.g., proximally and/or distally sliding the actuator in a proximal or distal direction), rotational movements (e.g., clockwise and/or counter clockwise rotation), and/or pressure actuated movements (e.g., button presses). In some aspects, the handle device 1600 can be configured to be mechanically actuated, electrically actuated, electromechanically actuated, and/or any other suitable actuating means. It should be understood that the above examples are not meant to be limiting, and any other suitable handle devices may be utilized with the transcatheter device 100 described herein.

The above-described configurations of the handle device 1600 are merely exemplary, and thus any other suitable configurations for actuating the transcatheter device 100 can be utilized. Furthermore, the handle device 1600 should not be construed as being configured to only actuate the above-mentioned components. Rather, the handle device 1600 can actuate any other suitable components of the transcatheter device 100, such as for example, valve apparatus 514, the expandable stent frame 502, etc. Furthermore, because the handle device 1600 is merely exemplary, any other suitable handle device may be utilized in actuating the transcatheter device 100.

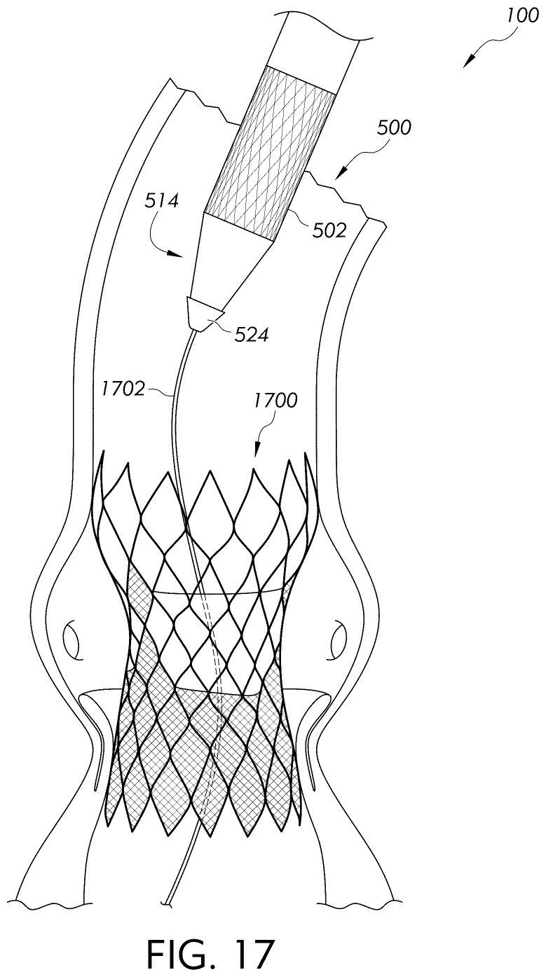

FIGS. 17-23 will describe a method of replacing a heart valve 1700 (e.g., a failing heart valve) with the heart valve prosthesis 500 with the understanding that similar or identical methods may be provided in the other embodiments of the disclosure. In some aspects, the heart valve 1700 can comprise the preexisting prosthetic heart valve 200. In some aspects, the heart valve 1700 can comprise a native heart valve, such as for example, a tricuspid valve, a pulmonary valve, a mitral valve, and/or an aortic valve.

Turing specifically to FIG. 17, in some aspects, the method can comprise distally advancing the heart valve prosthesis 500 comprising the expandable stent frame 502 in the contracted orientation into the heart valve 1700. In some aspects, distally advancing the heart valve prosthesis 500 can comprise distally advancing a transcatheter device 100 comprising the heart valve prosthesis 500 where at least the valve apparatus 514 of the heart valve prosthesis 500 can define a portion of the transcatheter device 100 (e.g., the distal tip 524 acts as the leading tip of the transcatheter device 100). In some such aspects, distally advancing the transcatheter device 100 can further comprise distally advancing the transcatheter device 100 along a guide wire 1702 and through a patient's vasculature. In some examples, the transcatheter device 100 can be distally advanced along the guide wire 1702 through an incision that is made at an anatomical location of the patient's vessel. For example, a transfemoral approach that involves making an incision in the groin and operates as a passageway for guiding the transcatheter device 100 to the heart valve 1700. In some aspects, the transcatheter device 100 can be distally advanced along the guide wire 1702 by way of the inner lumen 1406 extending through the transcatheter device 100 and the lumen 1402 extending through the distal apex 530 of the distal tip 524 of the heart valve prosthesis 500.

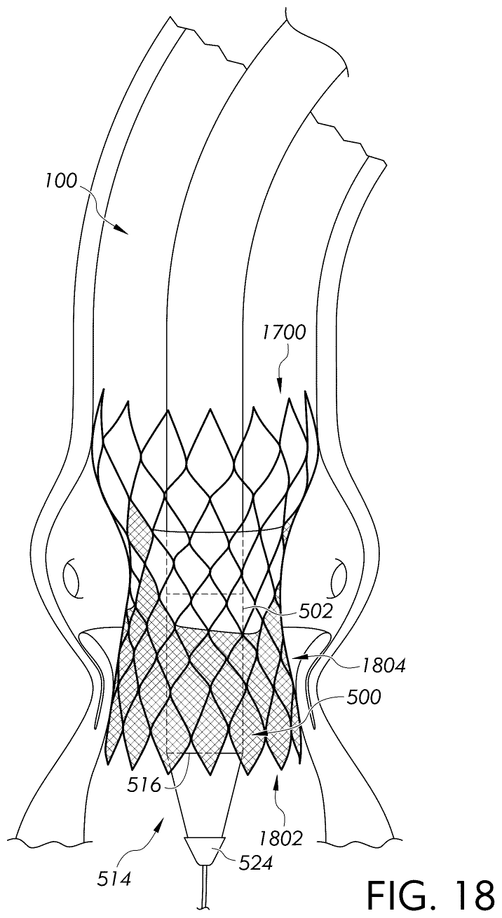

As shown in FIG. 18, in aspects, the method can comprise aligning a portion of the heart valve prosthesis 500 with a portion of the heart valve 1700. In some examples, aligning the portion of the heart valve prosthesis 500 with the portion of the heart valve 1700 can comprise aligning a portion of the expandable stent frame 502 of the heart valve prosthesis 500 with a distal portion 1804 (inflow portion) of the heart valve 1700. In some more particular examples, aligning the portion of the transcatheter device 100 can further comprise aligning the distal end 516 of the expandable stent frame 502 with a distal end 1802 of the heart valve 1700. In some examples where the heart valve 1700 is the preexisting prosthetic heart valve 200, the distal portion 1804 can be the distal portion 203. In some other examples where the heart valve 1700 is the native heart valve, the distal portion 1804 can be a distal portion of the native heart valve (e.g., tricuspid valve, aortic valve, etc.). In some aspects, aligning the portion of the heart valve prosthesis 500 can comprise aligning the valve apparatus 514 to extend past the distal end 1802 of the heart valve 1700. For example, when the valve apparatus 514 is deployed into a native aortic valve or a prior replacement aortic valve, at least a portion of the valve apparatus 514 may extend into the left ventricle. In some examples, a majority (e.g., over 50%) of the length of the leaflets may extend into the left ventricle, such as at least 75% of the length of the leaflets. In some aspects, aligning the portion of the heart valve prosthesis 500 can further comprise locating one or more radiopaque markers (not shown) on the heart valve prosthesis 500 and aligning the one or more radiopaque markers with the portion of the heart valve 1700. In some such aspects, the radiopaque markers can be any type of radiopaque marker and may be located on any portion of the transcatheter device 100 (e.g., the heart valve prosthesis 500, the outer shaft 102, the inner shaft 1404, etc.).

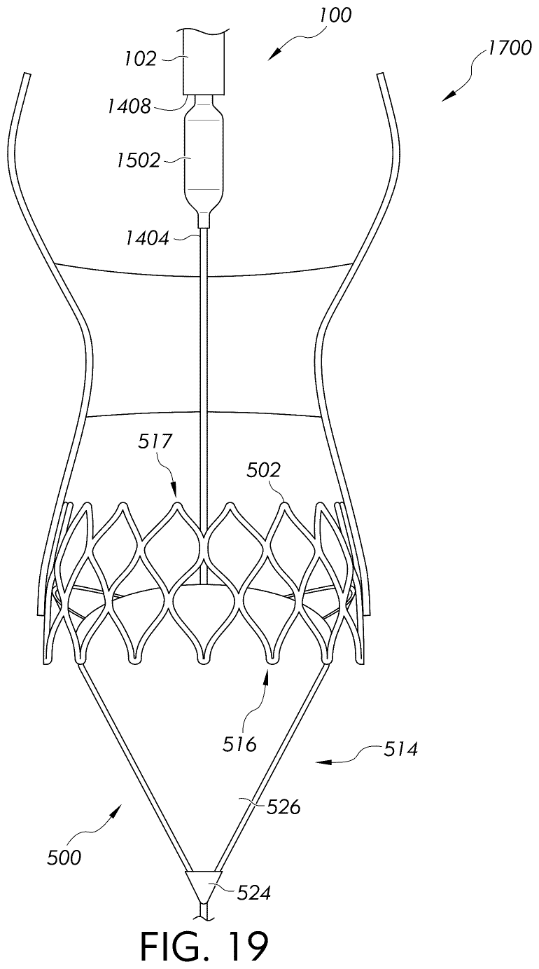

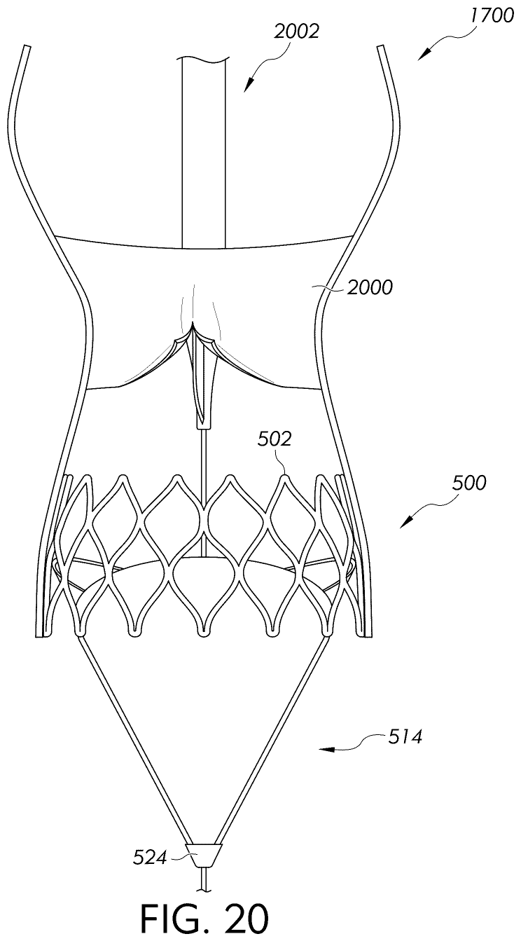

FIGS. 19-22 show a cutaway view of the heart valve 1700. In aspects, as illustrated in FIG. 19, the method can comprise radially expanding the expandable stent frame 502 from the contracted orientation to the expanded orientation. In some embodiments, radially expanding the expandable stent frame 502 can comprise proximally retracting the capsule 1412 of the transcatheter device 100. In some embodiments, radially expanding the expandable stent frame 502 can comprise inflating the expansion device 1502 of the transcatheter device 100. In some embodiments, radially expanding the expandable stent frame 502 can comprise proximally retracing the capsule 1412 of the transcatheter device 100, and then inflating the expansion device 1502 of the transcatheter device 100.

In some aspects shown in FIG. 19, the method can comprise removing the transcatheter device 100 from the heart valve 1902. For example, the method can comprise proximally retracting the transcatheter device 100 to remove the transcatheter device 100 from the heart valve 1700 and out of the patient's vasculature. In some examples, removing the transcatheter device 100 from the heart valve 1700 can first comprise proximally retracting the inner shaft 1404 and/or the expansion device 1502 (if provided), such as for example, proximally retracting the inner shaft 1404 and/or the expansion device 1502 to proximally retract to or past the distal end 1408 of the outer shaft 102 of the transcatheter device 100, and then further comprising removing the transcatheter device 100 from the heart valve 1700, and then out of the patient's vasculature. In other examples, removing the transcatheter device 100 from the heart valve 1700 can comprise proximally retracting the inner shaft 1404 and/or the expansion device 1502, thereby removing the transcatheter device 100 from the heart valve 1700. In this example, the transcatheter device 100 can then be proximally retracted from the patient's vasculature. In some examples, discussed hereinafter, the transcatheter device 100 may not be removed from the heart valve 1700 and be utilized to perform various additional and/or alternative functions.

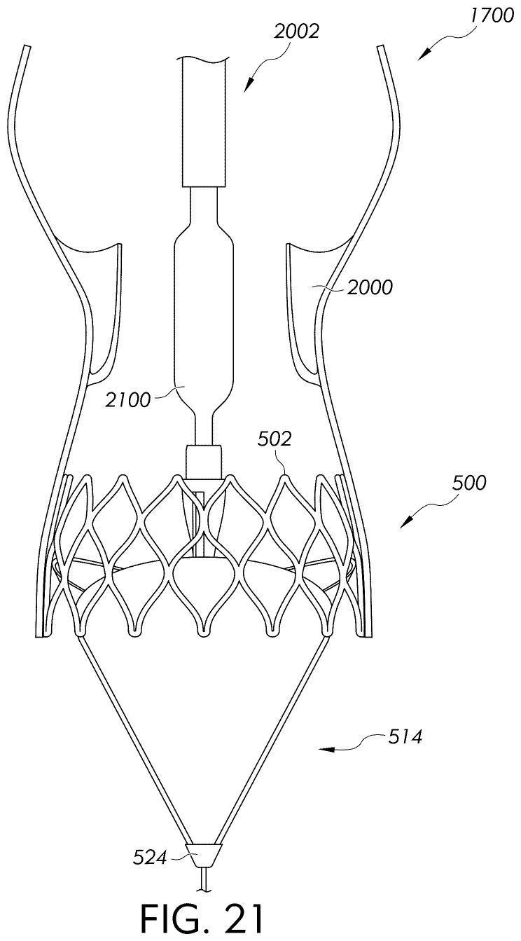

In aspects, as shown in FIG. 20, the method can further comprise lacerating at least one leaflet 2000 of the heart valve 1700. In some examples, the at least one leaflet 2000 can comprise prosthetic heart valve leaflets. For example, the prosthetic heart valve leaflets can comprise the prosthetic heart valve leaflets 204 of the preexisting prosthetic heart valve 200. In some examples, the at least one leaflet 2000 can comprise native heart valve leaflets. For example, the at least one leaflet 2000 can comprise tricuspid valve leaflets, pulmonary valve leaflets, mitral valve leaflets, and/or aortic valve leaflets. In some examples, lacerating at least one leaflet 2000 can comprise lacerating more than one leaflet, such as for example, lacerating two leaflets, three leaflets, etc. In some non-limiting examples, the at least one leaflet 2000 can be lacerated to bisect (e.g., lacerated to divide the at least one leaflet 2000 of the heart valve 1700 into two substantially equal parts) one or more of the at least one leaflet 2000. In some aspects, lacerating the at least one leaflet 2000 can comprise lacerating the at least one leaflet 2000 to create a first lacerated portion and a second laceration portion that are substantially equal in size. In other aspects, various other locations along one or more of the at least one leaflet 2000 can be lacerated such that the first lacerated portion and the second lacerated portion are not substantially equal. In other aspects, the at least one leaflet 2000 can be lacerated to form more than two lacerated pieces.

In some aspects, the at least one leaflet 2000 may be lacerated prior to inserting the transcatheter device 100. In aspects, the prosthetic heart valve leaflets may be lacerated while concurrently inserting the transcatheter device 100 into the patient and/or may be lacerated with a lacerating feature of the transcatheter device 100 (e.g., where the transcatheter device 100 has not been removed from the patient's vasculature). In some aspects, lacerating the at least one leaflet 2000 can comprise inserting a different transcatheter device 2002 (as shown) after removing the transcatheter device 100 from the patient's vasculature. Alternatively, in some aspects, the at least one leaflet 2000 may not be lacerated wherein the heart valve 1700 may be replaced without lacerating the at least one leaflet 2000. In some embodiments, the at least one leaflet 2000 may have been previously lacerated or resected in a prior procedure, and thus does not need to be lacerated. In some aspects, lacerating the at least one leaflet 2000 may comprise cutting, skiving, or splitting (e.g., by a cutting tool, such as a mechanical cutting tool (e.g., a blade), or a heating/energy cutting tool (e.g., RF electrode)). In some examples, lacerating the at least one leaflet 2000 may comprise lacerating more than one leaflet by varying the lengths of the lacerations of the leaflets, which can be done by lacerating from a free end of each leaflet towards the cusp of the heart valve 1700.

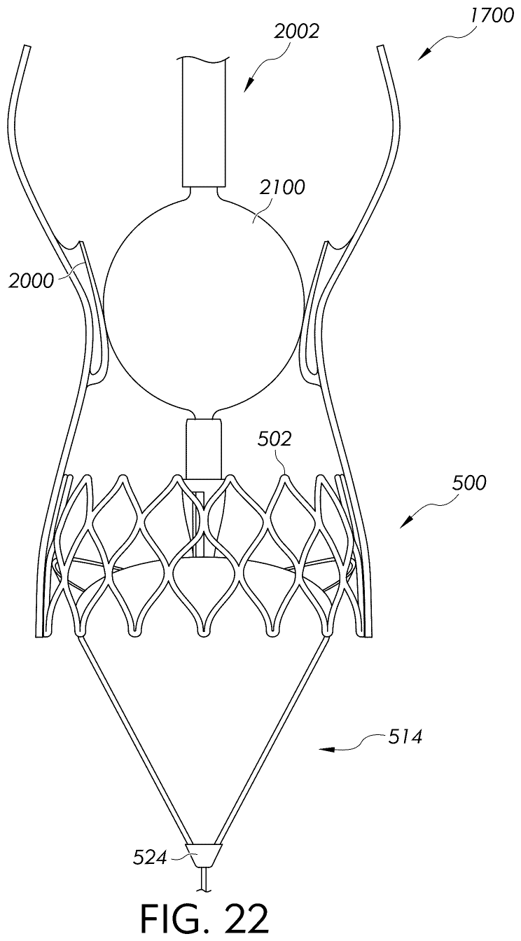

Turning to FIGS. 21-22, in some aspects, as shown best in FIG. 21, the method can comprise aligning an inflation device 2100 with the at least one leaflet 2000. In aspects, the method can further comprise inflating the inflation device 2100 (see FIG. 22) to reposition the at least one leaflet 2000 of the heart valve 1700. In aspects, the inflation device 2100 can be inflated after severing the at least one leaflet 2000 of the heart valve 1700. In further aspects, the inflation device 2100 can be inflated prior to or simultaneously with severing the at least one leaflet 2000 of the heart valve 1700. In some embodiments, the inflation device 2100 can comprise a portion of the different transcatheter device 2002, as shown in FIGS. 21-22. In some embodiments, the inflation device 2100 can comprise a portion of the transcatheter device 100 (e.g., where the transcatheter device 100 has not been removed from the patient's vasculature). In some embodiments, the inflation device 2100 can comprise the expansion device 1502 (if provided) of the transcatheter device 100. For example, after inflating the expansion device 1502 of the transcatheter device 100 to radially expand the expandable stent frame 502, the method can further comprise deflating the expansion device 1502, proximally retracting the expansion device 1502 to align the expansion device 1502 with the at least one leaflet 2000 of the heart valve 1700, and then reinflating the expansion device 1502 to reposition the at least one leaflet 2000 of the heart valve 1700. In some aspects, repositioning the at least one leaflet 2000 can comprise folding, collapsing, flattening, pushing, and/or bunching the at least one leaflet 2000 in any one of a distal direction, a radial direction and/or a proximal direction within the heart valve 1700.

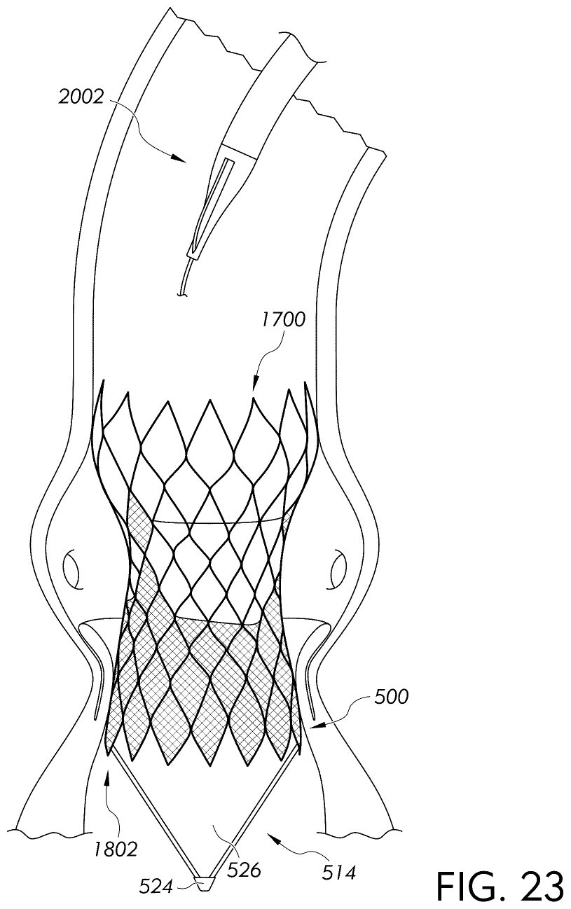

As shown in FIG. 23, the method can comprise removing the different transcatheter device 2002 from the heart valve 1700. For example, the method can comprise proximally retracting any or all portions of the different transcatheter device 2002 to remove any or all of the portions of the different transcatheter device 2002 from the heart valve 1700 and out of the patient's vasculature. It should be understood that in some embodiments discussed above where the transcatheter device 100 is used to perform various additional operations discussed previously (e.g., as opposed to the different transcatheter device 2002 performing the various additional operations), the transcatheter device 100 can then be removed as previously discussed with reference to FIG. 19.

In some aspects, the method can further comprise flowing blood around each leaflet of the plurality of leaflets 526 and through the opening 620 (see FIG. 6) of each leaflet of the plurality of leaflets 526. For example, when the heart valve prosthesis 500 has been deployed within the heart valve 1700 (e.g., the expandable stent frame 502 has been radially expanded) blood can flow around each leaflet and through the opening 620. In some examples, the heart valve prosthesis 500 can begin to function (e.g., blood can flow around each leaflet of the plurality of leaflets 526) prior to the heart valve prosthesis 500 being deployed (e.g., prior to the expandable stent frame 502 being radially expanded).

In aspects, by flowing blood around each leaflet of the plurality of leaflets 526 (as opposed to flowing blood through a central opening formed by converging leaflets) and by deploying the heart valve prosthesis 500 such that the valve apparatus 514 of the heart valve prosthesis 500 distally extends past the distal end 1802 of the heart valve 1700, an effective orifice area (e.g., the area formed by the opening 620) of the heart valve prosthesis 500 will be increased. In this way, the heart valve prosthesis 500 will not be impacted in situations where the heart valve 1700 has been impacted by stenosis (e.g., calcification of the heart valve resulting in a narrowing of the heart valve opening). Furthermore, in aspects, by deploying the heart valve prosthesis 500 such that the valve apparatus 514 of the heart valve prosthesis 500 distally extends past the distal end 1802 of the heart valve 1700, the heart valve prosthesis 500 will reduce the risk of coronary (e.g., the left coronary and/or the right coronary) obstruction, such as for example, sinus sequestration (e.g., where leaflets of a preexisting prosthetic heart valve sequestrate the sinuses).

In accordance with the disclosure, non-limiting aspects of the disclosure will now be described. Various combinations of the aspects can be provided in accordance with the disclosure.

Aspect 1. A heart valve prosthesis comprising an expandable stent frame configured to expand from a contracted orientation to an expanded orientation and comprising a proximal end portion and a distal end portion. The expandable stent frame extends from the proximal end portion to the distal end portion along an elongated axis of the expandable stent frame. The heart valve prosthesis further comprises a valve apparatus extending past a distal end of the distal end portion of the expandable stent frame. The valve apparatus comprising a plurality of commissure posts, each commissure post of the plurality of commissure posts comprising a first end portion attached to the distal end portion of the expandable stent frame, a second end portion attached to a distal tip of the valve apparatus at a distal end of the valve apparatus. The plurality of commissure posts converge together in a distal direction of the elongated axis towards the distal tip. The valve apparatus further comprises a plurality of leaflets each coupled to a corresponding pair of the plurality of commissure posts.

Aspect 2. The heart valve prosthesis of Aspect 1, wherein the plurality of leaflets comprise three leaflets.

Aspect 3. The heart valve prosthesis of any one of Aspects 1-2, wherein the plurality of leaflets each comprise a first edge attached along a first commissure post of the corresponding pair of commissure posts from the first end portion of the first commissure post to the second end portion of the first commissure post, and a second edge attached along a second commissure post of the corresponding pair of commissure posts from the first end portion of the second commissure post to the second end portion of the second commissure post.

Aspect 4. The heart valve prosthesis of any one of Aspects 1-3, wherein the plurality of leaflets each comprise a third edge configured to alternate between an inwardly bent configuration and an outwardly bent configuration.

Aspect 5. The heart valve prosthesis of Aspect 4, wherein the plurality of leaflets each comprise a convex surface facing the elongated axis in the inwardly bent configuration.

Aspect 6. The heart valve prosthesis of any one of Aspects 4-5, wherein the plurality of leaflets each comprise a convex surface facing away from the elongated axis in the outwardly bent configuration.

Aspect 7. The heart valve prosthesis of any one of Aspects 1-6, wherein the plurality of leaflets are configured to form a conical configuration.

Aspect 8. The heart valve prosthesis of any one of Aspects 1-7, wherein the plurality of leaflets each comprise a distal portion and a proximal portion, wherein a portion of the proximal portion of each leaflet of the plurality of leaflets extends proximally past the distal end of the expandable stent frame towards the proximal end portion of the expandable stent frame.

Aspect 9. The heart valve prosthesis of any one of Aspects 1-8, wherein the expandable stent frame comprises a self-expandable stent frame.

Aspect 10. The heart valve prosthesis of Aspect 9, wherein the expandable stent frame comprises a self-expanding material.

Aspect 11. The heart valve prosthesis of Aspect 10, wherein the self-expanding material comprises nitinol.

Aspect 12. The heart valve prosthesis of any one of Aspects 1-11, further comprising a hemostatic bumper coupled to the expandable stent frame.

Aspect 13. The heart valve prosthesis of Aspect 12, wherein the hemostatic bumper comprises a continuous band disposed around the entire elongated axis.

Aspect 14. The heart valve prosthesis of any one of Aspects 1-13, further comprising an outer skirt coupled to an outer surface of the expandable stent frame.

Aspect 15. The heart valve prosthesis of any one of Aspects 1-14, wherein the expandable stent frame further comprises a plurality of protrusions circumferentially spaced around the elongated axis.

Aspect 16. The heart valve prosthesis of any one of Aspects 1-15, wherein the distal tip further comprises a lumen extending through a distal apex of the distal tip.

Aspect 17. A transcatheter device comprising an outer shaft. The transcatheter device further comprises an inner shaft extending within the outer shaft and through a distal end of the outer shaft. The transcatheter device further comprises an expandable stent frame configured to expand from a contracted orientation to an expanded orientation. The expendable stent frame comprises a proximal end portion and a distal end portion. The expandable stent frame defines a lumen extending from the proximal end portion to the distal end portion along an elongated axis of the expandable stent frame. A distal segment of the inner shaft distally extends outside of the distal end of the outer shaft and through the lumen of the expandable stent frame. The transcatheter device further comprises a valve apparatus extending from a distal end of the distal end portion of the expandable stent frame. The valve apparatus comprises a plurality of commissure posts, each commissure post of the plurality of commissure posts comprising a first end portion attached to the distal end portion of the expandable stent frame, a second end portion attached to a distal tip of the valve apparatus at a distal end of the valve apparatus. The plurality of commissure posts converge together in a distal direction of the elongated axis towards the distal tip. The valve apparatus further comprises a plurality of leaflets each coupled to a corresponding pair of the plurality of commissure posts.

Aspect 18. The transcatheter device of Aspect 17, wherein the plurality of leaflets comprise three leaflets.

Aspect 19. The transcatheter device of any one of Aspects 17-18, wherein the plurality of leaflets each comprise a first edge attached along a first commissure post of the corresponding pair of commissure posts from the first end portion of the first commissure post to the second end portion of the first commissure post, and a second edge attached along a second commissure post of the corresponding pair of commissure posts from the first end portion of the second commissure post to the second end portion of the second commissure post.

Aspect 20. The transcatheter device of any one of Aspects 17-19, wherein the plurality of leaflets each comprise a third edge configured to alternate between an inwardly bent configuration and an outwardly bent configuration.

Aspect 21. The transcatheter device of Aspect 20, wherein the plurality of leaflets each comprise a convex surface facing the elongated axis in the inwardly bent configuration.

Aspect 22. The transcatheter device of any one of Aspects 20-21, wherein the plurality of leaflets each comprise a convex surface facing away from the elongated axis in the outwardly bent configuration.

Aspect 23. The transcatheter device of any one of Aspects 17-22, wherein the plurality of leaflets are configured to form a conical configuration.

Aspect 24. The transcatheter device of any one of Aspects 17-23, wherein the plurality of leaflets each comprise a distal portion and a proximal portion, wherein a portion of the proximal portion of each leaflet of the plurality of leaflets extends proximally past the distal end of the expandable stent frame towards the proximal end portion of the expandable stent frame.

Aspect 25. The transcatheter device of any one of Aspects 17-24, wherein the expandable stent frame comprises a self-expandable stent frame.

Aspect 26. The transcatheter device of Aspect 25, wherein the expandable stent frame comprises a self-expanding material.

Aspect 27. The transcatheter device of Aspect 26, wherein the self-expanding material comprises nitinol.

Aspect 28. The transcatheter device of any one of Aspects 17-24, further comprising a capsule attached to the distal end of the outer shaft, wherein the expandable stent frame is configured to be positioned within the capsule in the contracted orientation while the valve apparatus distally extends from a distal end of the capsule.

Aspect 29. The transcatheter device of any one of Aspects 17-24, further comprising an expansion device positioned within the lumen of the expandable stent frame and extending along the elongated axis.

Aspect 30. The transcatheter device of Aspect 29, wherein the expansion device comprises a balloon.

Aspect 31. The transcatheter device of any one of Aspects 17-30, further comprising a hemostatic bumper coupled to the expandable stent frame.

Aspect 32. The transcatheter device of Aspect 31, wherein the hemostatic bumper comprises a continuous band disposed around the entire elongated axis.

Aspect 33. The transcatheter device of any one of Aspects 17-32, further comprising an outer skirt coupled to an outer surface of the expandable stent frame.

Aspect 34. The transcatheter device of any one of Aspects 17-33, wherein the expandable stent frame further comprises a plurality of protrusions circumferentially spaced around the elongated axis.

Aspect 35. The transcatheter device of any one of Aspects 17-34, wherein the distal tip further comprises a lumen extending through a distal apex of the distal tip.

Aspect 36. A method of replacing a heart valve with a heart valve prosthesis. The heart valve prosthesis comprising an expandable stent frame comprising a proximal end portion and a distal end portion. The expandable stent frame extends from the proximal end portion to the distal end portion along an elongated axis of the expandable stent frame. The heart valve prosthesis further comprises a valve apparatus extending from a distal end of the distal end portion of the expandable stent frame. The valve apparatus comprises a plurality of commissure posts, each commissure post of the plurality of commissure posts comprising a first end portion attached to the distal end portion of the expandable stent frame, a second end portion attached to a distal tip of the valve apparatus at a distal end of the valve apparatus. The plurality of commissure posts converge together in a distal direction of the elongated axis towards the distal tip. The valve apparatus comprises a plurality of leaflets each coupled to a corresponding pair of the plurality of commissure posts. The method comprises distally advancing the heart valve prosthesis into the heart valve while the expandable stent frame is in a contracted orientation. The method further comprises aligning a portion of the heart valve prosthesis with a portion of the heart valve. The method then comprises radially expanding the expandable stent frame from the contracted orientation to an expanded orientation.

Aspect 37. The method of Aspect 36, further comprising lacerating at least one leaflet of the heart valve.

Aspect 38. The method of any one of Aspects 36-37, further comprising inflating an inflation device to reposition at least one leaflet of the heart valve.

Aspect 39. The method of Aspect 38, wherein the inflation device is inflated after lacerating at least one leaflet of the heart valve.

Aspect 40. The method of any one of Aspects 36-39, wherein radially expanding the expandable stent frame comprises proximally retracting a capsule of the transcatheter device.

Aspect 41. The method of any one of Aspects 36-39, wherein radially expanding the expandable stent frame comprises inflating an expansion device of the transcatheter device.

Aspect 42. The method of any one of Aspects 36-41, wherein aligning the portion of the heart valve prosthesis further comprises aligning a distal end of the expandable stent frame with a distal end of the heart valve.

Aspect 43. The method of any one of Aspects 36-42, further comprising alternating a bending of an edge of each leaflet of the plurality of leaflets between an inwardly bent configuration and an outwardly bent configuration.

Aspect 44. The method of Aspect 43, wherein the bending of the edge of each leaflet of the plurality of leaflets into the inwardly bent configuration defines a valve opening between the expandable stent frame and the corresponding leaflet, and the bending of the edge of each leaflet of the plurality of leaflets into the outwardly bent configuration closes the valve opening.

Aspect 45. The method of Aspect 44, wherein, when the edge of each leaflet of the plurality of leaflets is bent into the inwardly bent configuration, further comprising flowing blood around each leaflet of the plurality of leaflets and through the valve opening of each leaflet of the plurality of leaflets.

What is claimed is:

1. A heart valve prosthesis comprising:
an expandable stent frame configured to expand from a contracted orientation to an expanded orientation and comprising a proximal end portion and a distal end portion, wherein the expandable stent frame extends from the proximal end portion to the distal end portion along an elongated axis of the expandable stent frame; and
a valve apparatus extending past a distal end of the distal end portion of the expandable stent frame, the valve apparatus comprising:
a plurality of commissure posts, each commissure post of the plurality of commissure posts comprising a first end portion attached to the distal end portion of the expandable stent frame, a second end portion attached to a distal tip of the valve apparatus at a distal end of the valve apparatus, wherein the plurality of commissure posts converge together in a distal direction of the elongated axis towards the distal tip; and
a plurality of leaflets, each leaflet of the plurality of leaflets coupled to a corresponding pair of the plurality of commissure posts.

2. The heart valve prosthesis of claim 1, wherein each leaflet of the plurality of leaflets comprises a first edge attached along a first commissure post of the corresponding pair of commissure posts from the first end portion of the first commissure post to the second end portion of the first commissure post, and a second edge attached along a second commissure post of the corresponding pair of commissure posts from the first end portion of the second commissure post to the second end portion of the second commissure post.

3. The heart valve prosthesis of claim 1, wherein each leaflet of the plurality of leaflets comprises a third edge configured to alternate between an inwardly bent configuration and an outwardly bent configuration.

4. The heart valve prosthesis of claim 3, wherein each leaflet of the plurality of leaflets comprises a convex surface facing the elongated axis in the inwardly bent configuration.

5. The heart valve prosthesis of claim 3, wherein each leaflet of the plurality of leaflets comprises a convex surface facing away from the elongated axis in the outwardly bent configuration.

6. The heart valve prosthesis of claim 1, wherein the plurality of leaflets are configured to form a conical configuration.

7. A transcatheter device comprising:
an outer shaft;
an inner shaft extending within the outer shaft and through a distal end of the outer shaft;
an expandable stent frame configured to expand from a contracted orientation to an expanded orientation and comprising a proximal end portion and a distal end portion, wherein the expandable stent frame defines a lumen extending from the proximal end portion to the distal end portion along an elongated axis of the expandable stent frame, and a distal segment of the inner shaft distally extends outside of the distal end of the outer shaft and through the lumen of the expandable stent frame; and
a valve apparatus extending from a distal end of the distal end portion of the expandable stent frame, the valve apparatus comprising:
a plurality of commissure posts, each commissure post of the plurality of commissure posts comprising a first end portion attached to the distal end portion of the expandable stent frame, a second end portion attached to a distal tip of the valve apparatus at a distal end of the valve apparatus, wherein the plurality of commissure posts converge together in a distal direction of the elongated axis towards the distal tip; and
a plurality of leaflets, each leaflet of the plurality of leaflets coupled to a corresponding pair of the plurality of commissure posts.

8. The transcatheter device of claim 7, wherein each leaflet of the plurality of leaflets comprises a first edge attached along a first commissure post of the corresponding pair of commissure posts from the first end portion of the first commissure post to the second end portion of the first commissure post, and a second edge attached along a second commissure post of the corresponding pair of commissure posts from the first end portion of the second commissure post to the second end portion of the second commissure post.

9. The transcatheter device of claim 7, wherein each leaflet of the plurality of leaflets comprises a third edge configured to alternate between an inwardly bent configuration and an outwardly bent configuration.

10. The transcatheter device of claim 9, wherein each leaflet of the plurality of leaflets comprises a convex surface facing the elongated axis in the inwardly bent configuration.

11. The transcatheter device of claim 9, wherein each leaflet of the plurality of leaflets comprises a convex surface facing away from the elongated axis in the outwardly bent configuration.

12. The transcatheter device of claim 7, wherein the plurality of leaflets are configured to form a conical configuration.

13. A method of replacing a heart valve with a heart valve prosthesis, the heart valve prosthesis comprising an expandable stent frame comprising a proximal end portion and a distal end portion, wherein the expandable stent frame extends from the proximal end portion to the distal end portion along an elongated axis of the expandable stent frame;
and a valve apparatus extending from a distal end of the distal end portion of the expandable stent frame, the valve apparatus comprising: a plurality of commissure posts, each commissure post of the plurality of commissure posts comprising a first end portion attached to the distal end portion of the expandable stent frame, a second end portion attached to a distal tip of the valve apparatus at a distal end of the valve apparatus,
wherein the plurality of commissure posts converge together in a distal direction of the elongated axis towards the distal tip; and a plurality of leaflets, each leaflet of the plurality of leaflets coupled to a corresponding pair of the plurality of commissure posts, the method comprising:
distally advancing the heart valve prosthesis into the heart valve while the expandable stent frame is in a contracted orientation;
aligning a portion of the heart valve prosthesis with a portion of the heart valve; and
radially expanding the expandable stent frame from the contracted orientation to an expanded orientation.

14. The method of claim 13, further comprising lacerating at least one leaflet of the heart valve.

15. The method of claim 13, further comprising inflating an inflation device to reposition at least one leaflet of the heart valve.

16. The method of claim 13, wherein radially expanding the expandable stent frame comprises proximally retracting a capsule of the transcatheter device.

17. The method of claim 13, wherein radially expanding the expandable stent frame comprises inflating an expansion device of the transcatheter device.

18. The method of claim 13, further comprising alternating a bending of an edge of each leaflet of the plurality of leaflets between an inwardly bent configuration and an outwardly bent configuration.

19. The method of claim 18, wherein the bending of the edge of each leaflet of the plurality of leaflets into the inwardly bent configuration defines a valve opening between the expandable stent frame and the corresponding leaflet, and the bending the edge of each leaflet of the plurality of leaflets into the outwardly bent configuration closes the valve opening.

20. The method of claim 19, wherein, when the edge of each leaflet of the plurality of leaflets is bent into the inwardly bent configuration, further comprising flowing blood around each leaflet of the plurality of leaflets and through the valve opening of each leaflet of the plurality of leaflets.
